(19) United States
(12) Patent Application Publication
(10) Pub. No.: US 2024/0229161 A9
(43) Pub. Date: Jul. 11, 2024

(54) PREDICTING RESISTANCE TO TILAPIA LAKE VIRUS

(71) Applicants: THE UNIVERSITY COURT OF THE UNIVERSITY OF EDINBURGH, Edinburgh (GB); THE WORLDFISH CENTER - MALAYSIA OFFICE, Penang (MY)

(72) Inventors: Agustin Barria GONZALEZ, Edinburgh (GB); John BENZIE, Penang (MY); Ross HOUSTON, Midlothian (GB)

(21) Appl. No.: 18/547,784

(22) PCT Filed: Feb. 24, 2022
(86) PCT No.: PCT/GB2022/050506

§ 371 Date: Aug. 24, 2023

(30) Foreign Application Priority Data
Feb. 25, 2021 (GB) 2102696.8

(51) Int. Cl. C12Q 1/6888

(52) U.S. Cl. CPC C12Q 1/6888; C12Q 2600/124; C12Q 2600/156

(57) ABSTRACT

The present disclosure relates to methods of screening tilapia for increased genetic resistance to viral infection, such as Tilapia Lake Virus, as well as the use of these fish, which have been identified as having increased genetic resistance, in aquaculture breeding programs and production.

### FIELD

The present disclosure relates to methods of screening tilapia for increased genetic resistance to viral infection, such as Tilapia Lake Virus, as well as the use of these fish, which have been identified as having increased genetic resistance, in aquaculture breeding programs and production.

### BACKGROUND

Nile tilapia (Oreochromis niloticus) is the second most important farmed fish globally; worldwide production exceeded 4.2 million metric tons in 2016 and increasing annually. Outbreaks of infectious disease in fish grown in aquaculture is increasingly problematic, from an animal husbandry, animal welfare, environmental, economic, and food security perspective. Tilapia Lake Virus (TiLV) is one of the biggest threats to Nile tilapia aquaculture globally, and outbreaks can result in high levels of mortality in farmed stocks, from fingerlings to adults. Selective breeding to improve host resistance to the virus is a promising avenue to prevent or reduce mortalities, and the use of genomic tools can expedite this process.

### SUMMARY

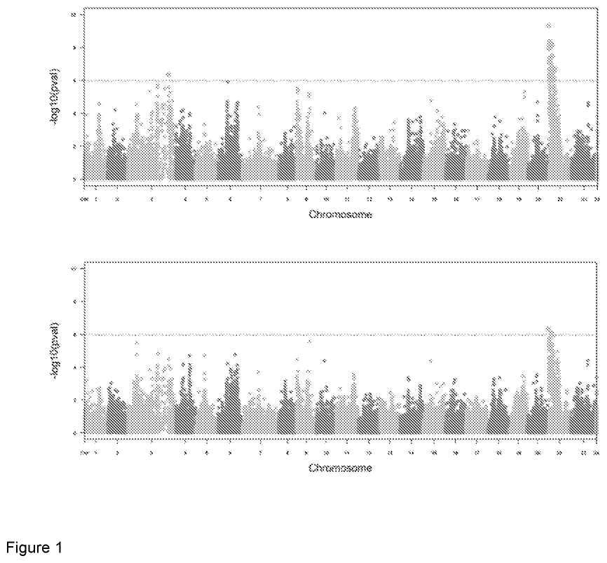

The present teaching is based on the identification a number of genetic alterations (polymorphisms), such as Single Nucleotide Polymorhpisms (SNPs), which are located in two Quantitative Trait Locus (QTLs) on the Oreochromis niloticus genome, specifically on chromosomes 22 (Oni22) and 3 (Oni3) (FIG. 1). These QTLs, and the polymorphisms found within them, are associated with host resistance to TiLV and may be of use in a breeding program to develop Nile tilapia strains with high levels of resistance to viruses, such as, TiLV.

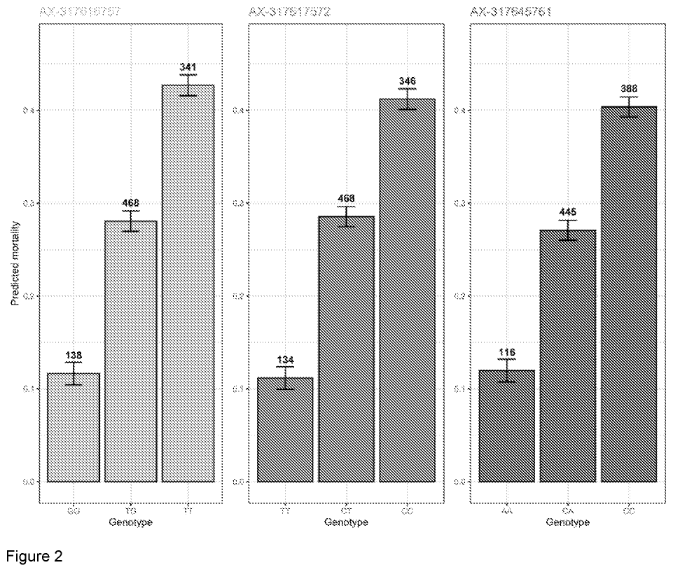

In the current disclosure, survival and mortality data from 1,821 fish was collected during a natural outbreak of TiLV, in a breeding population of the Genetically Improved Farmed Tilapia (GIFT) strain managed by WorldFish in Malaysia. A subset of these fish were genotyped using a 65 K Axiom® SNP array (Penaloza et al. 2020), and a genome-wide association study (GWAS) was performed using survival data from 950 fish and 48 K informative SNPs. The trait of host resistance was assessed as binary survival (i.e. 0=survivor, 1=mortality), and the number of days to death. Two significant QTLs were identified; on tilapia chromosomes Oni22 and Oni3 for the trait of binary survival. A single SNP on Oni22 showed the highest level of significance for both traits (P=4.51E-10 for binary survival, and 4.80E-07 for days to death), and both traits show a very high positive correlation. The average mortality rate of tilapia carrying two copies of the resistance allele at this SNP was 11%, compared to 43% for tilapia carrying two copies of the susceptibility allele, with heterozygous fish showing intermediate mortality levels (FIG. 2). Several candidate genes related with a viral infection process were identified to map close to these QTLs, including Igals17, trappc1, mf2, vps52, trm29 and cdc42. These results confirm that host resistance to TiLV is highly heritable (as previously shown in Barria et al. 2020), and crucially highlights genomic regions and genetic markers, which have a highly significant association with resistance.

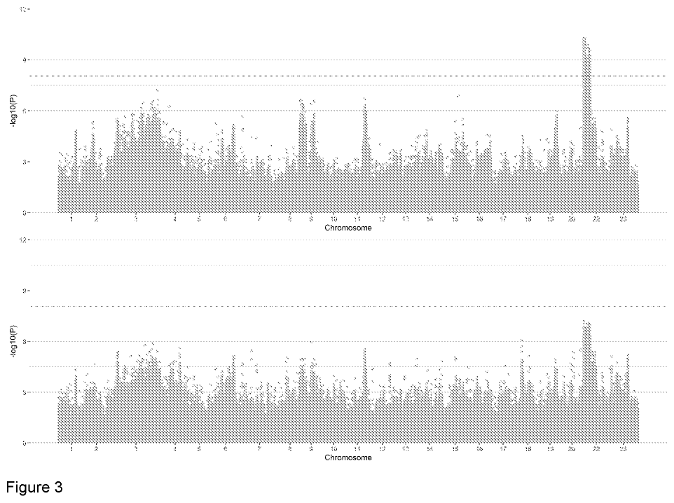

In parallel, 126 fish from generation 15, representing the parents of the fish outlined above, were sequenced using whole-genome sequencing (WGS). These data were used to impute the fish collected from the outbreak to full WGS data. Thus, we increased the number of SNPs, from 48 K to approximately 5 million, for all the assessed fish. After this, a new genome-wide association study was assessed, followed by a posterior fine-mapping narrowing the genomic intervals where the significant markers are located. A total of 564 bi-allelic markers were found to be significantly associated to binary survival (BS) (FIG. 3; Table 7). The highest level of significance, in line with the initial work, was found on the terminal end of Oni22 (P=4.70E-11). These significant markers segregates within a 10 Mb window in this chromosome and grouped in four different QTLs. This fine mapping confirms the results found with the SNP array data and provides more evidence about the genomic regions postulated to be associated with host resistance to TiLV. Additionally, we found that some of these significant markers are located within genes postulated as having an antiviral role during an infection process. Thus, genes as Igals17, vps52, hcmn1 and muc5ac were also confirmed and highlighted as genes likely involved in the host response during the viral infection process.

These genetic markers found either with the SNP array and with the WGS data can be applied to predict resistance of tilapia broodstock to viruses, such as TiLV, and therefore used in selective breeding programs and/or specific gene editing to improve genetic resistance and expedite the development of more resistant tilapia strains.

Furthermore, our findings highlights promising candidate genes with an antiviral role, to be involved in the host immune response, providing useful information about alleles associated with TiLV host resistance and therefore a target for potentially improving this trait by genome editing.

In a first aspect there is provided a method of determining whether or not a tilapia may display increased resistance to infection by a virus, the method comprising genotyping the tilapia in order to identify one or more nucleotide alterations within chromosomes 22 and/or 3 and determining whether or not the tilapia is resistant, or likely to display increased resistance to infection by the virus, or likely to have offspring which display increased resistance to infection by the virus.

In one embodiment, the method is conducted, in order to identify one or more nucleotide alterations within chromosome 22.

In one embodiment, the virus is a tilapinevirus of the family Amnoonviridae such as tilapia lake virus (TiLV), also known as syncytial hepatitis of tilapia (SHT).

Said nucleotide alteration(s) may be a substitution, deletion, inversion, addition or multiplication (e.g. duplication) of one or more nucleotides. In one embodiment, the nucleotide alteration is a SNP. A single-nucleotide polymorphism is a substitution of a single nucleotide at a specific position in the genome that is present in a sufficiently large fraction of the population (e.g. 1% or more). Typically, although not exclusively and without wishing to be bound by theory, the nucleotide alteration/SNP may result in a difference in RNA and/or protein expression levels of a gene or genes located in the identified region, or may result in alternative splicing and resulting expression of a gene or genes within the identified region. It may also result in a difference in protein amino acid sequence and/or protein structure. Alternatively, the SNP may be neutral and acting as a marker for a functional nucleotide alteration nearby in the genomic region.

In one embodiment the method comprises identifying if said one or more nucleotide alterations occur on both copies of the identified chromosomes and is considered homozygous for the alteration, or occurs on only one copy of the identified chromosome and is therefore considered as being heterozygous for the alteration. In one embodiment, the method identifies one or more homozygous nucleotide alterations.

Genetic analysis using the polymorphisms described herein, and others within the defined region of the QTL, may be of use in breeding programs in order to breed tilapia, which display increased resistance to viruses, such as TiLV, for example increased survival rate, and/or increased survival time. Accordingly, one embodiment of the disclosure provides a method of selecting a fish for a breeding program comprising testing fish for one or more nucleotide alterations in chromosomes 22 and/or 3, as described herein, such as, although not exclusively, SNPs listed in Tables 2 and/or 5 and/or 7 and selecting fish for the breeding program based on the presence or absence of the one or more nucleotide alterations.

In one embodiment, said one or more nucleotide alterations or SNPs are found in a region of approximately 10 Mb, between nucleotides 1 and 10,000,000 on chromosome 22 and/or in a region of 300 kb between nucleotides 71,697,333 and 71,997,333 on chromosome 3. Numbering according to NCBI and the O. niloticus genome (O_niloticus_UMD_NMBU, Genbank accession number GCA_001858045.3). The skilled addressee can readily identify corresponding or orthologous regions from other species of tilapia by performing cross-species sequence alignment and comparisons using the genome sequence data from this region.

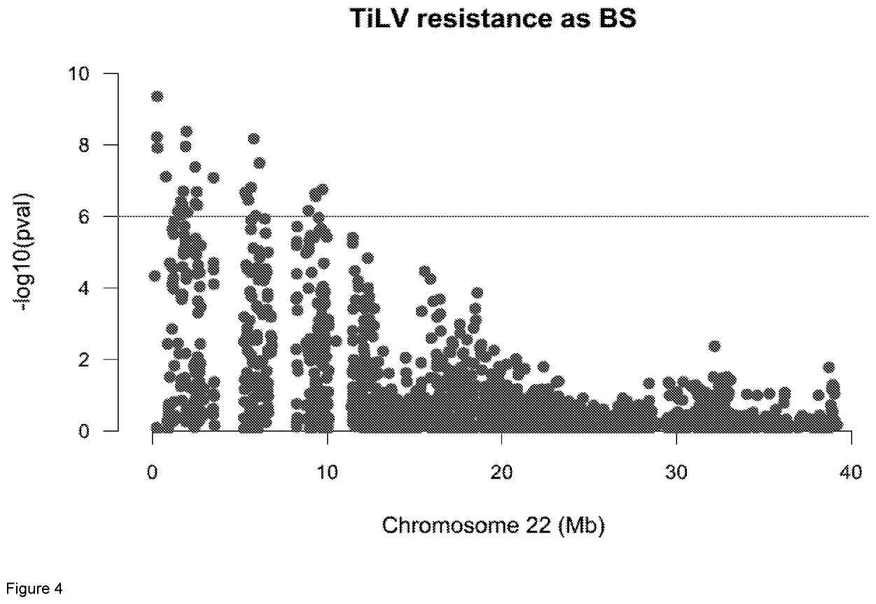

In accordance with this disclosure, resistance to infection may be, in one embodiment, correlated in terms of survival during an infection and, in another embodiment, an increase in survival time during an infection. In one embodiment both an increase in survival and an increase in survival time (days to death) may be taken into account. In one embodiment, only an increase in survival may be taken into account. In an alternative embodiment, only an increase in survival time (days to death) may be taken into account. When examining the pattern of the significant SNPs association with survival rate and survival time (FIGS. 4-6), it is clear that the most significant SNPs occur within a window of approximately 10 Mb on the proximal region of the chromosome. Therefore, when considering these parameters, said one or more nucleotide alterations or SNPs may be found in a region of approximately 10 Mb covering all the genome-wide significant SNPs on chromosome 22, between nucleotides 1 and 10,000,000 on chromosome 22 when considering association with increased survival. When considering both survival rate and survival time, the most significant SNPs fall within a region of approximately 6.2 Mb, between nucleotides 1 and 6,200,000 on chromosome 22 when correlating based on an increase in time to death. Finally, the region containing the three most highly significant SNPs for survival rate and most significant SNP for survival time is approximately 2 Mb, between nucleotides 1 and 2,000,000 on chromosome 22.

In some embodiments, said one or more nucleotide alterations or SNPs may only be found on chromosome 22 and the regions identified herein.

As well as SNPs which have been identified as being correlated with an increased survival and/or increased time to death as described herein (see Table 2), the present disclosure also extends to SNPs which are considered to be in linkage disequilibrium (LD) with the SNPs which have been identified through correlation with increased survival and/or increased time to death. LD is the non-random association of alleles at different loci in a given population. Loci are said to be in linkage disequilibrium when the frequency of association of their different alleles is higher or lower than what would be expected if the loci were independent and associated randomly. Association through LD can be determined by a variety of techniques known in the art. In accordance with the present disclosure, SNPs that were considered to be in LD with the SNPs identified by correlation with increased survival and/or increased time to death, where identified based on Pearson's squared correlation coefficient (r²). This statistic is widely used on aquaculture and terrestrial species for LD measurement, mainly due to it being less sensitive to bias and more appropriate for biallelic markers, such as SNPs. Thus, the present disclosure extends to further markers with an r²≥0.6 (such as 0.7, 0.8, 0.9 or 1), with the significant SNPs associated with host resistance to TiLV, identified herein and located within a 1 Mb window flanking the significant SNPs were considered to be in LD with the identified SNPs and are encompassed by this disclosure. Exemplary SNPs which are in such LD with the SNPs identified in Table 2, are identified in Table 5

In one embodiment, said one or more SNPs comprises or consists of one or more SNPs identified in Tables 2, 5 and/or 7. In one embodiment, said one or more SNPs comprises or consists of one or more of the following SNPs:
AX-317616757 and AX-317647630;
AX-317616757, AX-317617572 and AX-317645761; and
AX-317718855, or combinations thereof, optionally in combination with one or more other SNPs identified in Tables 2, 5 and/or 7.

In one embodiment, said one or more SNPs comprises or consists of:
AX-317616757, optionally in combination with one or more other SNPs identified in Tables 2, 5 and/or 7.

As well as the specific SNPs, which have been identified, genes which are within 500 kb of each SNP, have been identified. As such, in one embodiment, the method may further comprise determining, whether or not, expression of one or more genes within 500 kb (upstream and downstream) of a SNP identified in Tables 2, 5 and/or 7 has been altered, for example, increased or decreased. Specific SNPs and genes which are located within 500 kb of each SNP are identified in Tables 5 and 6.

A fine-mapping analysis using whole genome sequencing data confirms the genomic regions located on chromosome 22 as significantly associated with host resistance to Tilapia Lake Virus when defined as survival rate. Thus, 564 significant SNPs were found in the same 10 Mb region size covering all the genome-wide significant SNPs on chromosome 22 (Table 7). These significant SNPs can be categorized into four different QTLs based on their location along the 10 Mb significant genomic region located in chromosome 22. The first comprises between nucleotide 1 and 354,572 and contains half of the identified significant SNPs. The second region has a size of 2.3 Mb including the nucleotides between 1.3 Mb and 3.6 Mb. The third region includes nucleotides between 5.19 Mb to 6.4. The last genomic regions where significant SNPs were found refers a 1 Mb region size including the nucleotides from 8.2 Mb to 9.2 Mb. When considering only the top 25 most significant SNPs, 22 of them fall within a region of 340 Kb, between nucleotides 1 and 340,795 on chromosome 22.

In one embodiment, the said one or more nucleotide alterations may be found in a region of approximately 360 kb, between 1 and 360,000 on chromosome 22 which contains half of the significant SNPs for survival rate, including some of the most significant, found through the fine-mapping analysis.

The fine-mapping highlights some of the genes included in Table 3 and previously suggested as candidate genes likely involved with host resistance. We now identified significant SNPs that are located within some of these genes, generating a nonsynonymous mutation, thereby a change in the amino acid conformation of the protein product and therefore a likely change in its structure and/or activity. These genes are underlined on Table 3 and includes Igals17, vps52, hcmn1 and muc5ac.

In one embodiment, the said one or more nucleotide alterations generate a nonsynonymous mutation in any of the genes listed in Table 3. In one embodiment, the said significant SNPs may be located within the genes Igals17, vps52, hcmn1 and muc5ac.

The present disclosure may relate to any species of tilapia, for example Oreochromis or Sarotherodon species. Examples of commercially important species include Nile tilapia, Blue tilapia (Oreochromis auteus) and Mozambique tilapia (Oreochromis mossambicus), blackchin tilapia (Sarotherodon melanotheron), spotted tilapia (Pelmatolapia marae), and redbelly tilapia (Coptodon zillii). In one embodiment the present disclosure relates to Nile tilapia. As mentioned above, although the specific chromosomal locations identified herein are in respect of O. niloticus, it is straightforward for the skilled reader to identify corresponding regions from other tilapia species.

A fish that is determined to have increased resistance to virus infection according to this disclosure is more likely than normal to produce offspring that have a higher than normal chance of having increased resistance to viral infection. Consequently, in a further aspect of the disclosure, there is provided a method of selecting a tilapia for use as broodstock, wherein the tilapia is selected, based on a method as described herein above, to have increased resistance to viral infection. Conveniently, host resistance to TiLV is not related to the sex of the tilapia. Therefore, both male and female fish which are identified as having increased resistance to virus infection may be selected for use as broodstock.

Conversely, a tilapia predicted by the method as described herein above, as not having increased resistance to viral infection, would not be selected as broodstock. In accordance with the above, there is provided a population of tilapia, which has been obtained from at least one male and at least one female tilapia, which has been identified by a method as described herein to have increased resistance to virus infection

In a further embodiment, the SNPs of the present disclosure may be used in Marker Assisted Selection (MAS), wherein tilapia enrolled in a breeding program are checked in accordance with a method as described hereinabove, for the presence or absence of one or more identified SNPs. This could take the form of a diagnostic genetic test comprising the genetic markers in the QTL region, as identified herein. For example, tilapia having one or more SNPs as identified herein as increasing resistance to virus infection, may be placed into a breeding program in order to select for offspring that also carry that SNP. Accordingly, the SNPs can be used to non-lethally screen potential broodstock for increased resistance to virus infection. For example, a piece of a fin tissue can be obtained from a fish from a breeding program, and DNA can be extracted and analyzed to determine whether one or more nucleotide alterations in the identified QTL regions, such as the SNPs as identified herein is present. If the one or more nucleotide alteration/SNPs associated with resistance to virus infection are present, that fish would be desirable to include in a breeding program.

The term “allele” means any one of a series of two or more different gene sequences that occupy the same position or locus on a chromosome.

The term “genotype” means the specification of an allelic composition at one or more loci within an individual organism. In the case of diploid organisms such as tilapia, there are two alleles at each locus; a diploid genotype is said to be homozygous when the alleles are the same, and heterozygous when the alleles are different.

As used herein “genotyping” refers to determining the genotype of an organism at a particular locus, such as a SNP.

As used herein, “quantitative trait locus” or “QTL” refers to a genetic locus that contributes, at least in part, to the phenotype of an organism for a trait that can be numerically measured.

A person skilled in the art will appreciate that a number of methods can be used to determine the presence of the genetic alterations/SNPs identified in the present disclosure. For example a variety of techniques are known in the art for detecting a gene alteration/SNP within a sample, including genotyping, microarrays (also known as SNP arrays, or SNP chips), Restriction Fragment Length Polymorphism, Southern Blots, SSCP, dHPLC, single nucleotide primer extension, allele-specific hybridization, allele-specific primer extension, oligonucleotide ligation assay, and invasive signal amplification, Matrix-assisted laser desorption/ionization time-of-flight (MALDI-TOF) mass spectrometry, and Fluorescence polarization (FP).

Accordingly, the gene alterations/SNPs are detected by genotyping. Methods of genotyping are well known in the art. In one method, primers flanking the nucleotide alteration/SNP are selected and used to amplify the region comprising the SNP. The amplified region is then sequenced using DNA sequencing techniques known in the art and analyzed for the presence of the nucleotide alteration/SNP.

In another embodiment, the method of determining a nucleotide alteration/SNP comprises using a probe. For example, in one embodiment an amplified region comprising the nucleotide alteration/SNP is hybridized using a composition comprising a probe specific for the nucleotide alteration/SNP under stringent hybridization conditions.

Thus, the disclosure further teaches isolated nucleic acids that bind to nucleotide alterations/SNPs at high stringency that are used as probes to determine the presence of the gene alteration/SNP. In a particular embodiment, the nucleic acids are labeled with a detectable marker. The marker or label is typically capable of producing, either directly or indirectly, a detectable signal. For example, the label may be radio-opaque or a radioisotope, such as ³H, ¹⁴C, ³²p, ³⁵S, ¹²³I, ¹²⁵I, ¹³¹I; a fluorescent (fluorophore) or chemiluminescent (chromophore) compound, such as fluorescein isothiocyanate, rhodamine or luciferin; an enzyme, such as alkaline phosphatase, beta-galactosidase or horseradish peroxidase; an imaging agent; or a metal ion.

The term “probe” refers to a nucleic acid sequence that will hybridize to a nucleic acid target sequence. In one example, the probe hybridises to a sequence comprising a specific nucleotide alteration/SNP or its complement, under stringent conditions, but will not to the corresponding alternative allele or its complement. The length of probe depends on the hybridization conditions and the sequences of the probe and nucleic acid target sequence. In one embodiment, the probe is an oligonucleotide of 8-50 nucleotides in length, such as, 8-10, 8-15, 11-15, 11-20, 16-20, 16-25, 21-25, or 15-40 nucleotides in length.

In a further embodiment, there is provided a kit for use in one or more of the methods described herein, the kit comprising one or more probes for hybridising to said one or more nucleotide alterations within chromosomes 22 and/or 3, as identified herein. In one embodiment, the kit only comprise probes for hybridising to said one or more nucleotide alterations within chromosomes 22 and/or 3. That is the kits does not comprise probes capable of specifically hybridizing under stringent conditions to any other chromosome. The probes in the kit may comprise or consist of 1, 2, 3, 4, 5, 10, 15, 20, 25, 30, 40, 50, 75, 100, 500, or 1000 probes which are designed to specifically hybridise to said one or more nucleotide alterations within chromosomes 22 and/or 3, as identified herein.

A kit could take any variety of forms. In one embodiment the kit may comprise a substrate upon which said probe(s) are bound or otherwise attached to. The probes may be provided in a form of array, where individual probes of bound/adhered to specific and discernable locations on the substrate, so as to easily facilitate with identifying which probes bind to test nucleic acid.

The skilled addressee is well aware of other components such as reagents, buffers, nucleotides etc, which may be included in a kit.

By “stringent conditions” it is meant that conditions are selected which promote selective hybridization between two complementary nucleic acid molecules in solution. Hybridization may occur to all or a portion of a nucleic acid sequence molecule. Those skilled in the art will recognize that the stability of a nucleic acid duplex, or hybrids, is determined by the Tm, which in sodium containing buffers is a function of the sodium ion concentration and temperature (Tm=81.5X−16.6 (Log 10 [Na+])+0.41 (% (G+C)−600/l), or similar equation).

Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tm, for example if nucleic acid molecules are sought that have a >95% identity, the final wash temperature will be reduced by about 5° C. Based on these considerations those skilled in the art will be able to readily select appropriate hybridization conditions. In preferred embodiments, stringent hybridization conditions are selected. By way of example the following conditions may be employed to achieve stringent hybridization: hybridization at 5× sodium chloride/sodium citrate (SSC)/5×Denhardt's solution/1.0% SDS at Tm−5° C. for 15 minutes based on the above equation, followed by a wash of 0.2×SSC/0.1% SDS at 60° C. It is understood, however, that equivalent stringencies may be achieved using alternative buffers, salts and temperatures. Additional guidance regarding hybridization conditions may be found in: Current Protocols in Molecular Biology, John Wiley & Sons, N. Y., 1989, 6.3.1-6.3.6 and in: Sambrook et al., Molecular Cloning, a Laboratory Manual, Cold Spring Harbor Laboratory Press, 1989, Vol. 3. [0072] Nucleic acid sequences that are primers are useful to amplify DNA or RNA sequences containing a nucleotide alteration/SNP of the present disclosure. Accordingly, in one teaching, the disclosure provides a composition comprising at least one isolated nucleic acid sequence that is a specific probe or primer able to hybridise and/or amplify a sequence comprising a nucleotide alteration/SNP identified in table 3 and/or 7. A person skilled in the art would understand how to identify and test probes/primers that are useful for detecting/amplifying sequences containing the nucleotide alterations/SNPs identified herein.

In a further embodiment, the SNPs are detected using a primer extension assay. Briefly, an interrogation primer is hybridised to the sequence nucleotides immediately upstream of the nucleotide alteration/SNP nucleotide. A DNA polymerase then extends the hybridized interrogation primer by adding a base that is complementary to the nucleotide alteration/SNP. The primer sequence containing the incorporated base is then detected using methods known in the art. In one embodiment, the added base is a fluorescently labeled nucleotide. In another embodiment, the added base is a hapten-labelled nucleotide recognized by antibodies.

Such detection techniques known in the art include microarrays, hybridization assays, molecular beacons, Dynamic allele-specific hybridization (DASH) and/or combinations of these.

The nucleotide alterations/SNPs described herein are optionally detected using restriction enzymes. For example, amplified products can be digested with a restriction enzyme that specifically recognizes sequence comprising one of the nucleotide alteration/SNP alleles, but does not recognize the other allele. In one embodiment PCR is used to amplify DNA comprising a nucleotide alteration/SNP, amplified PCR products are subjected to restriction enzyme digestion under suitable conditions and restriction products are assessed. If for example a specific nucleotide alteration/SNP allele corresponds to a sequence digested by the restriction enzyme, digestion is indicative of detecting that particular nucleotide alteration/SNP allele. Restriction products may be assayed electrophoretically as is common is the art.

Nucleotide alteration/SNP alleles can also be detected by a variety of other methods known in the art. For example, PCR and RT-PCR and primers flanking the nucleotide alteration/SNP can be employed to amplify sequences and transcripts respectively in a sample comprising DNA (for PCR) or RNA (for RT-PCR). The amplified products are optionally sequenced to determine which of the nucleotide alteration/SNP alleles is present in the sample.

In one embodiment, the disclosure includes isolated nucleic acid molecules that selectively hybridize under stringent conditions to one of the SNPs identified in Tables 2 and/or 5 and/or Table 7. A further embodiment includes an isolated nucleic acid molecule that selectively hybridizes to a nucleic acid comprising a SNP allele or its complement. The phrase “specifically hybridizes to a SNP allele or its complement” means that under the same conditions, the isolated nucleic acid sequence will preferentially hybridize to one of the SNPs alleles or its complement, as compared to the other allele. The term “hybridize” refers to the sequence specific non-covalent binding interaction with a complementary nucleic acid. In a preferred embodiment, the hybridization is under high stringency conditions.

### DETAILED DESCRIPTION

The present disclosure will now be further described by way of example and with reference to the Figures, which show:

FIG. 1. Manhattan plot for resistance to Tilapia Lake Virus (TILV) in a Nile tilapia (Oreochromis niloticus) breeding population using SNP array data. Manhattan plot of GWAS for host resistance, as binary survival (top) and as time to death (bottom), to TiLV. On the y axis is the −log 10(P-value). Horizontal dashed red line shows the genome-wide significance threshold. Oni24 represent SNPs with unknown chromosome location.

FIG. 2. Predicted mortality values for host resistance to Tilapia Lake Virus in a Nile tilapia breeding population. Host resistance as binary survival predictive values for each genotype of the SNP with stronger genome-wide association. The bars on yellow, light blue and green shows the predicted values for the top three SNPs located on Oni22. The bars show the standard error. Numbers above the bars indicates the number of fish with the specific genotype.

FIG. 3. Ultra high resolution manhattan plot for resistance to Tilapia Lake Virus (TILV) in a Nile tilapia (Oreochromis niloticus) breeding population. Manhattan plot of GWAS for host resistance, as binary survival (top) and as time to death (bottom), to TiLV. On the y axis is the −log 10(P-value). Horizontal dashed red line shows the genome-wide significance threshold.

FIG. 4. Regional manhattan plot, on Oni22, for resistance to Tilapia Lake Virus (TILV) as binary survival (BS). Regional manhattan plot of GWAS for host resistance, as binary survival. On the y axis is the −log 10(P-value). Horizontal red line shows the genome-wide significance threshold, whereas each dot represents a single SNP, highlighting the 10 Mb region of interest.

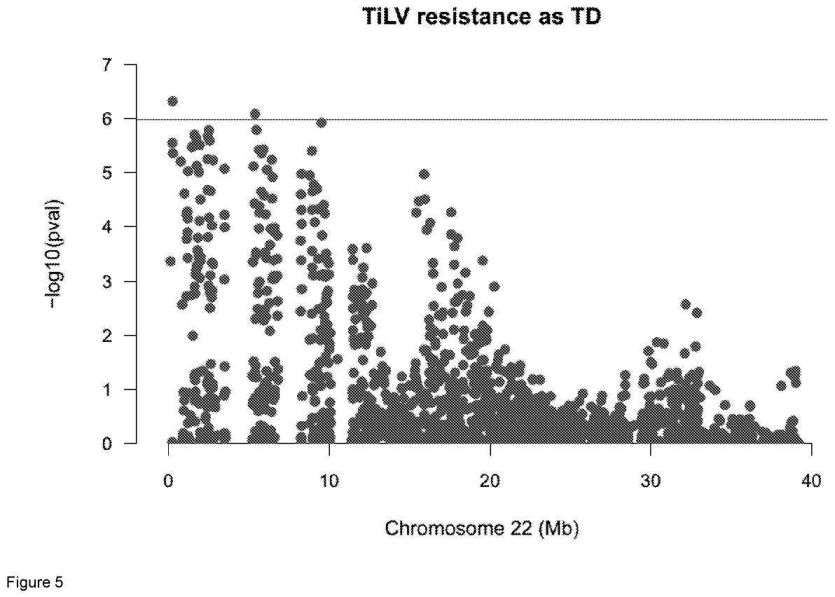

FIG. 5. Regional manhattan plot, on Oni22, for resistance to Tilapia Lake Virus (TILV) as time to death (TD). Regional manhattan plot of GWAS for host resistance, as time to death. On the y axis is the −log 10(P-value). Horizontal red line shows the genome-wide significance threshold, whereas each dot represents a single SNP.

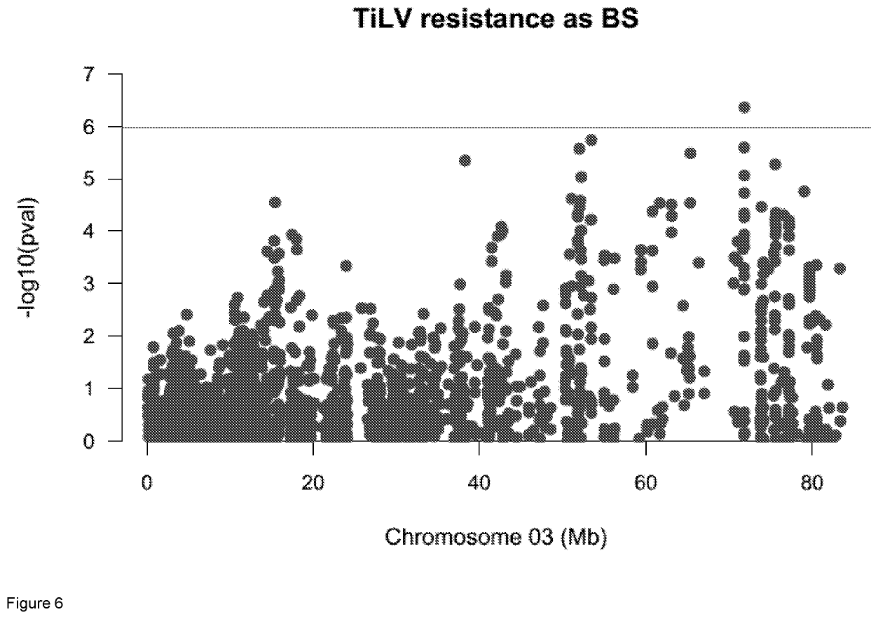

FIG. 6. Regional manhattan plot, on Oni3, for resistance to Tilapia Lake Virus (TILV) as binary survival (BS). Regional manhattan plot of GWAS for host resistance, as binary survival. On the y axis is the −log 10(P-value). Horizontal red line shows the genome-wide significance threshold, whereas each dot represents a single SNP.

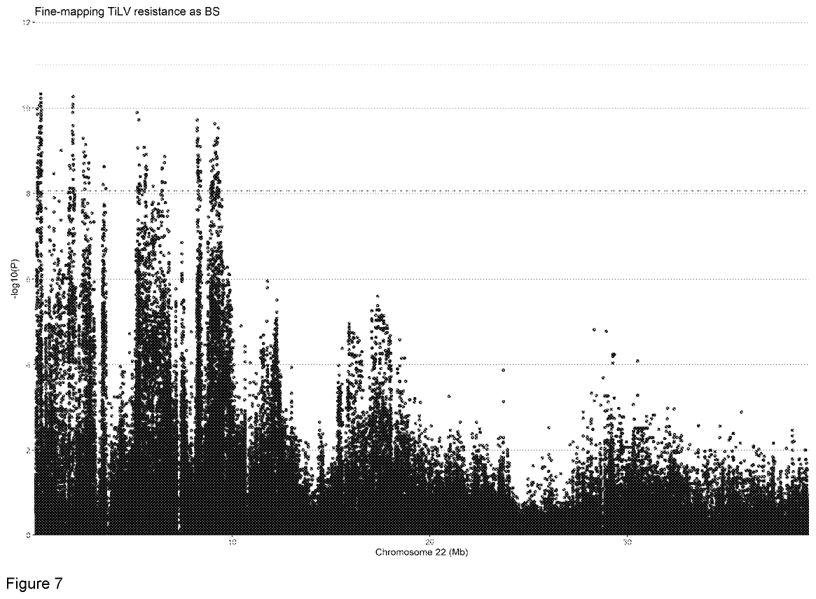

FIG. 7. Ultra high density regional manhattan plot, on Oni22, for resistance to Tilapia Lake Virus (TiLV) as binary survival (BS). Regional manhattan plot of GWAS for host resistance, as binary survival. On the y axis is the −log 10(P-value). Horizontal red line shows the genome-wide significance threshold, whereas each dot represents a single SNP, highlighting the 10 Mb region of interest.

### MATERIALS AND METHODS

Nile Tilapia Population

The population sample used in this study belong to a GIFT Nile tilapia breeding program which has been selected for growth rate for 15 generations. The breeding nucleus is based in Jittra, Malaysia and is managed by WorldFish. This population sample consisted of 124 nuclear families, produced by crossing 124 dams and 115 sires. Pedigree data for these fish included records of approximately 86,000 fish. Each fish from the current generation was tagged using a Passive-Integrated Transponder (PIT tag) at an average weight of 4.97 g, which corresponded to an average age of 110.5 days. At typical harvest weight, fish were transferred to a single pond where a natural TiLV outbreak was observed.

Natural Field Outbreak

After transfer of the fish to the single pond, a natural field outbreak of TiLV was observed (in February 2018). Mortalities were collected and sampled daily, and once the mortality levels had returned to baseline all remaining fish in the pond were euthanized (using 400 mg/i clove oil) and sampled. A total of 1,821 fish were classified as survivors or mortalities, and phenotypic sex was identified for all fish. On average, each full sibling family included 14 fish (which ranged from 2 to 21). Clinical signs of TiLV were observed throughout the outbreak, and a qPCR assay was performed to identify the presence of TiLV in the spleen of 39 fish. A sample of mortalities were randomly selected to also perform necropsy assays to further confirm TiLV as the cause of the mortalities. A caudal fin sample was taken from survivors and mortalities, kept in 95% ethanol, and stored at −80° C. until further analysis.

TILV Resistance Phenotype

Host resistance to TiLV was defined as binary survival (BS) (i.e. dead/alive at the end of the natural field outbreak) and as time to death (TD). In case of BS, the survivors and mortalities were treated as 0 and 1, respectively. Resistance as TD was treated as a continuous trait, with values ranging from 1 (day of the first observed mortality) to 18 or 19 conditional on the sampling day (this corresponded to the dates at which the mortalities had returned to baseline and the remaining fish in the pond were euthanized).

Genotyping

Total DNA from fin clips of 1,325 fish, including 195 parents and 1,130 offspring, was extracted by using a modified salt-extraction protocol proposed by Aljanabi and Martinez, (1997), with modifications as described on Taslima et al., (2016). The extracted DNA was genotyped using an Axiom® SNP array developed by our team which contains ˜65 K SNP markers dispersed throughout the genome of Nile tilapia (Penaloza et al. 2020). The genotyping was performed by Identigen (Dublin, Ireland). The raw data from the genotyping (CEL files) were imported to the Axiom analysis Suite v 4.0.3.3 software for genotype calling and quality control (QC). A total of 47 samples with a dish quality control (DQC) and quality control call rate (QC CR)<0.82 and <0.93, respectively, were excluded for subsequent analyses. Thus, 187 parents (96%) and 1,091 offspring (97%) passes the affymertix quality control. Regarding the number of SNPs, 54 K (78%) were identified as with PolyHighResolution and were considered for further analyses. Subsequently, a second QC step was applied using Plink v 1.09 (Purcell et al., 2007). With an average call rate of 99%, all fish surpassed the genotype call rate (>0.95). The SNPs with a minor allele frequency (MAF)<0.05, call rate <0.95 and with significant deviation from Hardy-Weinberg Equilibrium (HWE) (p<1×10⁻⁶) were excluded from further analyses. Thus, 94% of the SNPs (50,710 out 53,811) passed all the QCs, with most of them being removed due low MAF (˜2 K SNPs). Furthermore, using trio information, the resulting data set was tested for putative Mendelian errors in any fish and SNPs. Thus, a total of 217 fish and 3 K SNPs were excluded for subsequent analyses due a Mendelian error rate >5%. Finally, the remaining data set comprises 1,061 fish and 47, 915 SNPs. The former includes data from 950 offspring and 111 parents. Because phenotypic data from the TiLV field outbreak was measured only on the offspring, the genomic data of these individuals were used for the genome-wide association study.

Imputation Analysis

Illumina paired-end whole genome sequencing (WGS) was performed on 126 fish belonging to generation 15th (G15) from WorldFish at approximately 15-fold coverage on average. These fish are the parents of the animals collected after the natural outbreak of TiLV. The reads generated after the sequencing step were mapped to the Nile tilapia reference genome (GCA_001858054.3), followed by a variant calling analysis by using BCFtool. Once Indels and monomorphic SNPs were removed, a total of 16,286,750 bi-allelic SNPs were obtained. Then, a second quality control step was performed. Thus, we retained the markers that meet the following criteria for the further analyses: i) an average read depth >=2000 and <=3500, ii) mapping quality >30, iii) quality score >30 and iv) must be anchorage to chromosomes, remaining a total of 7,271,637 SNPs.

The 48 K SNP discovered in the fish collected from the TiLV outbreak were imputed to the 7M SNPs found in their parents. This was performed chromosome by chromosome, by using the Fimpute3.0 software. After imputation, a total of 5,723,303 SNPs with a MAF higher than 1% were filtered for further analyses.

Estimation of Genetic Parameters

The heritability for BS and TD was estimated using the genomic-relationship matrix (GRM) with the genome-wide complex trait analysis (GCTA) software v. 1.92.2 (Yang et al., 2011a).

All SNPs surpassing the QC were used to create the GRM. The GRM was then used to estimate the narrow-sense heritability. For both resistance definitions, the following linear model was used:

y=μ+Xb+Zu+e (1)

Where y is the vector of phenotypes (BS or TD records), p is the population mean, b is the vector of fixed effects (sex as fixed effect, and weight and age at harvest as covariates), u is the vector of the additive genetic effects, and X and Z are incidences matrices. The following distributions were assumed; u˜N(0, Gσᵤ²) and e·N(0, Iσₑ²). Where σᵤ² and σₑ² are the additive genetic and residual variance, respectively, G is the genomic relationship matrix and I is the identity matrix. Heritability was estimated through univariate analyses and as the ratio of the additive genetic variance to the phenotypic variance. Genetic correlation was estimated as the ratio of the covariance between BS and TD to the square root of the product of the variance of BS and TD.

Genome-Wide Association Study

To identify SNPs associated with TiLV resistance (both BS and TD traits), for the SNP array and WGS data, a mixed linear model leaving-one-chromosome-out (LOCO) approach was applied using the GCTA v. 1.92.2 software. This approach estimates the genomic relationship matrix (GRM) between individuals by removing the SNPs located in the tested chromosome and including SNPs from all the other chromosomes. Thus, the effect of markers from the chromosome of the specific SNP being tested is not included twice in the model. Subsequently, the GRM allows correction for population structure, which can cause spurious associations in GWAS. The model used for the GWAS was identical the model described in (1). However, single marker effects were included as variables in the model. For a SNP to be considered significant at the genome-wide level, it had to surpass the genome-wide Bonferroni-corrected significance threshold for multiple testing of 0.05/47,915 and 0.05/5.723,303 for SNP array and WGS data, respectively. This multiple test correction is considered very stringent (Johnson et al., 2010), which reduces the likelihood of any false positive association. To quantify the level of inflation of the obtained P-values compared with those expected, lambda (λ) was computed as the median of the quantile χ² distribution of the obtained P-values/0.455. For practical reasons, SNPs not placed in chromosomes in the reference genome assembly (O_niloticus_UMD_NMBU, Genbank accession number GCA_001858045.3, Conte et al., 2019), were assigned as Oni24. GWAS results were plotted by using the package “CMplot” in R.

Candidate Genes

Based on the SNP array genome-wide association results, putative candidate genes associated with host resistance to TiLV were identified within a 1 Mb windows size (500 Kb upstream and downstream) flanking the significantly associated SNPs, again using the Nile tilapia reference genome assembly (Genbank accession number GCA_001858045.3). For the genes identified through the fine-mapping analysis, only those that were affected by a nonsynonymous mutation were considered as likely associated with host resistance.

SNP Variances

Following the GWAS, the top three SNPs significantly associated with BS and/or TD on each of the two significant chromosomes were tested for the estimation of the additive and dominance effect, by using ASReml v. 4.1.0 (Gilmour et al., 2015). Thus, additive (a) and dominance (d) effect were estimated as follow: a=(AA−BB)/2 and d=AB−[AA+BB/2] where AA, AB and BB are the predicted trait value for each genotype. The proportion of genetic variance explained for each of the selected SNPs were estimated as [2pq(a+d(q−p))2]/VA, where p and q are the frequencies of the SNP alleles, and VA is the total additive genetic variance explained by the model when none SNP is fitted.

Results

Field Outbreak

Throughout the outbreak, clinical signs related with an infection process by TiLV were observed. These were confirmed by a qualified veterinarian, and subsequently TiLV was identified in a random sample of fish by a qPCR assay. Total cumulative mortality in the outbreak was 39.6%. For more details about outbreak data please refer to Barria et al., (2020).

Estimation of Variance Components

Moderate to high heritability values of 0.38±0.05 and 0.69±0.09 were estimated for BS on the observed and underlying scale, respectively, whereas a lower value was estimated for TD (0.22 t 0.05). A very high genetic correlation was found between both TiLV resistance definitions (0.97±0.02). Estimated additive genetic, residual and phenotypic variance for BS and TD using the genomic data are shown in Table 1. Using the imputed WGS data, similar moderate to high heritabilities were found for both traits, with estimates close to 0.65 and 0.20 for BS and TD, respectively.

Genome-Wide Association Study

In case of the SNP array data set, several SNPs were identified that exceeded the genome-wide significance Bonferroni threshold (−log₁₀(0.05/47,915)=5.98) for BS and TD (FIG. 1). A total of 29 SNPs have a P-value significantly associated with BS ranging from 9.65E-07 to 4.5E-10. From these markers, one single SNP is located in Oni03 (AX-317718855; P-value=4.37×10⁻⁰⁷, FIG. 7), while all the others are located in Oni22 (Table 2). In case of TD, two SNPs located on Oni22 surpassed this significance threshold. Interestingly, for both resistance definitions, the most significant association was found for the same SNP (AX-317616757, located at a position of 255,104 bp) with a P-value of 4.5×10⁻⁰⁷ and 4.8×10⁻⁰⁷, for BS and TD, respectively (Table 2). All the SNPs located in Oni22 which were significantly associated with BS are within a genomic region of approximately 9.4 Mb of size (FIG. 4). However, this QTL size is reduced to ˜1.7 Mb when only the tops three SNPs are taking into account (AX-317617572 and AX-317645761 located on 1,939,192 and 239,073 bp). Furthermore, the latter could potentially be split into two different QTLs by considering AX-317616757 (255,105 bp) and AX-317645761 (239,073 bp) as one QTL, and AX-317617572 (1,939,192 bp) as a second QTL. In the case of TD, the two markers that surpassed the Bonferroni significant threshold are located within a genomic region of ˜5.1 Mb (FIG. 5). The estimated inflation factor (A) for BS and TD is 1.19 and 1.11, suggesting a relatively good concordance between the observed P-values and the theoretical statistic distribution.

The complete list of genes flanking the SNPs with the strongest association, within each chromosome, for host resistance to TiLV (4 SNPs for BS and 2 SNPs for TD) and the area of QTL region where these genes were identified, and are shown in Table 3. A number of interesting candidate genes were found to map within the QTL region which have previously been found to be related to host response to a viral infection. For the main QTL on Oni22 the genes mf2 (E3 ubiquitin-protein ligase RING2-A), vps52 (VPS52 subunit of GARP complex), cdc42 (cell division control protein 42 homolog) were identified. For the secondary QTL on Oni3, the zbed1 (zinc finger BED-type containing) also known as dref (DNA replication-related element binding factor), trappc1 (trafficking protein particle complex 1) and psmb6 (proteasome subunit beta type-6) were identified. The other SNP found to be associated with TD and BS (AX-317647630) is flanked by two genes belonging to the tripartite motif family, trim21 and trim29.

As expected, the genome-wide fine-mapping analysis showed an increased number of markers surpassing the significance threshold, reaching up to 564 SNPs significantly associated with BS (FIG. 3). In agreement with the results discussed above, all of these markers are located within a 10 Mb region on chromosome 22, with a peak of significance on the proximal end of the chromosome, indicating the key role thesei genomic regions play for host resistance to Tilapia Lake Virus. The fine-mapping analysis allowed to identify significant SNPs located within gene sequences in the Nile tilapia reference genome. From all the genes identified with a significant SNPs within it sequences, we focused on those genes affected by a nonsynonymous mutation. Thus, the significant SNPs generates a change in an amino acid, the basic structure of the protein, which eventually could affect the structure and/or activity of the protein. Therefore, these genes are more likely to be involved in the host response to TiLV. The genes affected by this mutation are reduced to four genes, and are those underlined in Table 3, as were previously suggested as candidate genes.

Effect Size of the Significant QTL

The Minor Allele Frequency (MAF), additive and dominance effect, and proportion of additive genetic variance for the top three most significant SNPs related with host resistance, within each chromosome, are shown in Table 4. The estimated MAF for these SNPs range from 0.21 to 0.39 and from 0.11 to 0.39 in case of those associated with BS and TD, respectively. The minor allele is associated with resistance to TiLV. The three most significant SNPs located in Oni22 have a substitution effect on TiLV mortality proportion ranging from 0.16 to 0.14 (Table 4 and FIG. 2). In the case of the SNP located in Oni03 (AX-317718855), the equivalent allele substitution effect is 0.07. In case of TD, the allele substitution effect was −1.37 days (towards an early day of death) with a P-value of 3.12E-06. The proportion of genetic variance explained by the SNPs shown in Table 4 ranged from 0.06 to 0.14. As expected, the most significant SNP (AX-317616757).

The results highlight that the genetic architecture of host resistance to TiLV is ‘oligogenic’, with one highly significant QTL on Oni22, and a further significant QTL on Oni3. The predicted mortality rate for the most significant SNPs linked to these QTL is shown in FIG. 2. Based on the field outbreak data collected, the predicted mortality for homozygous fish for the resistance-associated allele for the most significant SNP (AX-317616757) is 0.11, contrasted to the mortality for homozygous fish for the susceptibility associated allele of 0.43. Therefore, the predicted difference in mortality between alternate homozygous fish at this single significant QTL is 32%, which can be placed in context by considering that the overall mortality rate in the outbreak was ˜40%.

### REFERENCES

Aljanabi, S. M., and Martinez, I. (1997). Universal and rapid salt-extraction of high quality genomic DNA for PCR-based techniques. Nucleic Acids Reseach 25.
Barria, A., Trinh, T. Q., Mahmuddin, M., Benzie, J. A. H., Chadag, V. M., and Houston, R. D. (2020). Genetic parameters for resistance to Tilapia Lake Virus (TiLV) in Nile tilapia (Oreochromis niloticus). Aquaculture 522.
Conte, M. A., Joshi, R., Moore, E. C., Nandamuri, S. P., Gammerdinger, W. J., Roberts, R. B., et al. (2019). Chromosome-scale assemblies reveal the structural evolution of African cichlid genomes. Gigascience 8, 1-20.
Gilmour, A. R., Gogel, B. J., Cullis, B. R., Welham, S. J., and Thompson, R. (2015). ASReml User Guide.
Johnson, R. C., Nelson, G. W., Troyer, J. L., Lautenberger, J. A., Kessing, B. D., Winkler, C. A., et al. (2010). Accounting for multiple comparisons in a genome-wide association study (GWAS). BMC Genomics 11, 724. doi:10.1186/1471-2164-11-724.
Purcell, S., Neale, B., Todd-brown, K., Thomas, L., Ferreira, M. A. R., Bender, D., et al. (2007).
PLINK: A Tool Set for Whole-Genome Association and Population-Based Linkage Analyses. Am. J. Hum. Genet. 81, 559-575.
Peñaloza C., Robledo D., Barria A., Trinh T., Mahmuddin M., Wiener P., Benzie J. and Houston R. D. Development and validation of an open access SNP array for Nile tilapia (Oreochromis niloticus). G3: Genes, Genomes, Genetics Aug. 1, 2020 vol. 10 no. 8 2777-2785.
Taslima, K., Davie, A., McAndrew, B. J., and Penman, D. J. (2016). DNA sampling from mucus in the Nile tilapia, Oreochromis niloticus: minimally invasive sampling for aquaculture-related genetics research. Aquac. Res. 47, 4032-4037. doi:10.1111/are.12809.
Yang, J., Weedon, M. N., Purcell, S., Lettre, G., Estrada, K., Willer, C. J., et al. (2011b). Genomic inflation factors under polygenic inheritance. Eur. J. Hum. Genet. 19, 807-812. doi:10.1038/ejhg.2011.39.

TABLE 1
Genetic parameters for host resistance to TiLV in a
Nile tilapia (Oreochromis niloticus) breeding population.
Standard error are shown inside brackets.

| Parameters^{b} | BS^{a} | TD^{a} |
|---|---|---|
| σₐ² | 0.09(0.02) | 5.96(1.47) |
| σₑ² | 0.14(0.01) | 21.6(1.33) |
| σₚ² | 0.22(0.01) | 27.57(1.39) |
| h² | 0.38(0.05) | 0.22(0.05) |

| | |
|---|---|
| r_{g} | −0.97(0.02) |

^{a}Host resistance definition: BS = Binary survival; TD = Time to death
^{b}Genetic parameters and standard error: σₐ² = additive genetic variance; σₑ² = error variance;
h² = narrow-sense estimated heritability;
r_{g} genetic correlation.

TABLE 2
Significant SNPs associated with TiLV resistance as binary
survival (BS) and time to death (TD) in a Nile tilapia
(Oreochromis nilotocus) breeding population.

| | | | | | |
|---|---|---|---|---|---|
| | | | | Minor/ | Resistance |
| | | | | Major | allele |
| SNP | Oni^{a} | Bp^{b} | p-value^{c} | allele^{d} | frequency |

TD

| | | | | | |
|---|---|---|---|---|---|
| AX-317616757 | 22 | 255104 | 4.80xE−07 | G/T | 0.39 |
| AX-317647630 | 22 | 5379664 | 8.20xE−07 | G/A | 0.11 |

BS

| | | | | | |
|---|---|---|---|---|---|
| AX-317616757 | 22 | 255104 | 4.51E−10 | G/T | 0.39 |
| AX-317617572 | 22 | 1939192 | 4.32E−09 | T/C | 0.39 |
| AX-317645761 | 22 | 239073 | 6.17E−09 | A/C | 0.36 |
| AX-317619074 | 22 | 5785936 | 6.86E−09 | G/A | 0.37 |
| AX-317617531 | 22 | 1888302 | 1.13E−08 | G/A | 0.47 |
| AX-317616778 | 22 | 276593 | 1.24E−08 | A/G | 0.56 |
| AX-317648645 | 22 | 6115097 | 3.30E−08 | A/G | 0.46 |
| AX-317617852 | 22 | 2432164 | 4.23E−08 | T/G | 0.40 |
| AX-317616808 | 22 | 760642 | 7.96E−08 | T/C | 0.48 |
| AX-317647368 | 22 | 3478052 | 8.47E−08 | A/G | 0.48 |
| AX-317647975 | 22 | 5629013 | 1.60E−08 | G/T | 0.16 |
| AX-317621349 | 22 | 9718148 | 1.81E−08 | G/A | 0.72 |
| AX-317617390 | 22 | 1758060 | 2.02E−08 | C/T | 0.42 |
| AX-317647016 | 22 | 2521284 | 2.13E−08 | A/G | 0.14 |
| AX-317618485 | 22 | 5289967 | 2.22E−08 | T/C | 0.39 |
| AX-317649969 | 22 | 9299308 | 2.38E−08 | A/G | 0.58 |
| AX-317647630 | 22 | 5379664 | 2.70E−08 | G/A | 0.11 |
| AX-317620863 | 22 | 9336674 | 2.81E−08 | C/T | 0.45 |
| AX-317647753 | 22 | 5479097 | 3.56E−08 | C/A | 0.11 |
| AX-317617254 | 22 | 1616487 | 3.92E−08 | C/T | 0.11 |
| AX-317718855 | 03 | 71847333 | 4.37E−07 | C/T^{e} | 0.24 |
| AX-317646938 | 22 | 2445275 | 4.45E−07 | A/G | 0.10 |
| AX-317646411 | 22 | 1714252 | 4.95E−07 | A/G | 0.10 |
| AX-317617977 | 22 | 2555673 | 4.97E−07 | C/T | 0.14 |
| AX-317620317 | 22 | 8916395 | 6.99E−07 | C/T | 0.13 |
| AX-317063470 | 22 | 1441242 | 7.27E−07 | A/G | 0.10 |
| AX-317646673 | 22 | 1988041 | 7.86E−07 | A/G | 0.29 |
| AX-317617288 | 22 | 1660924 | 9.54E−07 | T/G | 0.11 |
| AX-317648270 | 22 | 5898003 | 9.65E−07 | G/A | 0.12 |

^{a}Number of chromosome on the Oreochromis niloticus genome.
^{b}Position of the SNP in the chromosome, in base pairs.
^{c}P value of the SNP for the genome-wide association study for host resistance to TiLV.
^{d}In bold is the allele conferring the resistant phenotype.
^{e}The resistant genotype is the heterozygous.

TABLE 3
Genes flanking the most important genome-wide associated
SNPs within each chromosome for TiLV resistance.

| | | QTL region^{b} | | |
|---|---|---|---|---|
| Oni^{a} | Trait | Left position | Right position | Gene names |
| 03 | BS | 71,347,333 | 72,347,333 | zbed1^{c}, kcnab1, trappc1, nlrc3, psmb6, pigr, |
| | | | | nlrc3, mrc1, ephb4, fcgr2b, agp4, btnl2, |
| | | | | btnl10 |
| 22 | BS and | 1 | 755,104 | zhx1, lgals17^{d}, vps52, hmcn1, senp1, |
| | TD | | | muc5ac, half, rnf2, atad2, rps18, ptk7 |
| 22 | BS | 1,439,192 | 2,439,192 | zbed1, zscan2, tmem65, sema6d, scgn, |
| | | | | tatdn1, pomc, NADH, mtss1, grik5, fibcd1, |
| | | | | carmil1, rnf139, ceacam5, atx2 |
| 22 | BS | 1 | 739,073 | zhx1, lgals17, vps52, hmcn1, senp1, muc5ac, |
| | | | | half, rnf2, atad2, rps18, ptk7 |
| 22 | TD | 4,879,664 | 5,879,664 | cacgn7, cacgn6, cacgn4, vamp2, styk1, |
| | | | | trim29, tpi1, glut1, phc1, iffo2, gnb3, gapdh, |
| | | | | gtan, eno2, g2e3, trim21, cox6b1, chd4, |
| | | | | clcn1, cdc42, cd209e, clec6a |

^{a}Number of chromosome on the Oreochromis niloticus genome.
^{b}QTL region size was defined as 500 kb upstream and downstream the position of the SNP.
^{c}In bold the name of the genes with a role known to be involved in a viral infection process.
^{d}Genes underlined represents those with a nonsynonymous mutation, identified through the fine mapping analysis

TABLE 4
Summary statistics for the most significant genome-wide associated
SNPs within each chromosome for host resistance to TiLV.

| | | | | | |
|---|---|---|---|---|---|
| Oni^{a} | SNP | BP^{b} | a^{d} | Pval (a)^{e} | Vg^{f} |

Binary survival (BS)

| | | | | | |
|---|---|---|---|---|---|
| 22 | AX-317616757 | 0.25 | 0.16 | 2.45E−09 | 0.12 |
| 22 | AX-317617572 | 1.93 | 0.15 | 1.19E−08 | 0.10 |
| 22 | AX-317645761 | 0.23 | 0.14 | 1.90E−07 | 0.09 |
| 3 | AX-317718855 | 71.8 | 0.07 | 8.42E−02 | 0.06 |

Time to death (TD)

| | | | | | |
|---|---|---|---|---|---|
| 22 | AX-317616757 | 0.25 | −1.37 | 3.12E−06 | 0.14 |
| 22 | AX-317647630 | 5.37 | −1.97 | 5.45E−02 | 0.13 |

^{a}Number of chromosome on the Oreochromis niloticus genome.
^{b}Position of the SNP in the chromosome, in million base pairs.
^{c} additive genetic effect.
^{d}P value of the Student's t test distribution for the genetic effect.
^{e}Proportion of the genetic variance explained by the SNP.

TABLE 5
SNPs that shows a linkage disequilibrium (r²) higher
than 0.6 with respect to the most significant SNPs
associated with resistance to TiLV as BS or TD

| | | | | Major | |
|---|---|---|---|---|---|
| | | | | Allele/ | |
| | | | | Minor | |
| SNP | Chromosome | Bp | LD (r2) | Allele | Group |
| AX-317718814 | 3 | 71798319 | 0.631949 | G/A | 1 |
| AX-317701294 | 3 | 71835821 | 0.651731 | G/A | 1 |
| AX-317718855 | 3 | 71847333 | 1 | T/C | 1 |
| AX-317645761 | 22 | 239073 | 1 | C/A | 2 |
| AX-317616757 | 22 | 255104 | 0.791926 | T/G | 2 |
| AX-317616778 | 22 | 276593 | 1 | G/A | 2 |
| AX-317616808 | 22 | 760642 | 0.753679 | T/C | 2 |
| AX-317063468 | 22 | 1188968 | 1 | G/A | 3 |
| AX-317617134 | 22 | 1220867 | 0.930815 | A/C | 3 |
| AX-317063470 | 22 | 1441242 | 1 | G/A | 3 |
| AX-317617254 | 22 | 1616487 | 1 | T/C | 3 |
| AX-317617288 | 22 | 1660924 | 1 | G/T | 3 |
| AX-317646411 | 22 | 1714252 | 1 | G/A | 3 |
| AX-317617390 | 22 | 1758060 | 0.652366 | T/C | 3 |
| AX-317063478 | 22 | 1769725 | 0.831137 | T/A | 3 |
| AX-317036586 | 22 | 1788209 | 0.7967 | T/C | 3 |
| AX-317617423 | 22 | 1798900 | 0.826191 | G/A | 3 |
| AX-317617452 | 22 | 1819001 | 0.831137 | A/G | 3 |
| AX-317646557 | 22 | 1869386 | 0.867667 | G/A | 3 |
| AX-317617516 | 22 | 1878932 | 0.886976 | G/T | 3 |
| AX-317617531 | 22 | 1888302 | 0.600416 | A/G | 3 |
| AX-317617572 | 22 | 1939192 | 0.773432 | C/T | 3 |
| AX-317646635 | 22 | 1949770 | 0.834921 | C/A | 3 |
| AX-317646663 | 22 | 1979297 | 0.870039 | C/T | 3 |
| AX-317646673 | 22 | 1988041 | 0.705086 | G/A | 3 |
| AX-317617620 | 22 | 1996173 | 0.870039 | G/A | 3 |
| AX-317617852 | 22 | 2432164 | 0.900781 | G/T | 3 |
| AX-317617862 | 22 | 2437589 | 0.784468 | C/T | 3 |
| AX-317646938 | 22 | 2445275 | 1 | G/A | 3 |
| AX-317063485 | 22 | 2488042 | 0.883968 | C/T | 3 |
| AX-317647016 | 22 | 2521284 | 0.650087 | G/A | 3 |
| AX-317617977 | 22 | 2555673 | 0.650087 | T/C | 3 |
| AX-317618014 | 22 | 2585167 | 0.65962 | T/G | 3 |
| AX-317647368 | 22 | 3478052 | 1 | G/A | 4 |
| AX-317618485 | 22 | 5289967 | 0.818822 | C/T | 5 |
| AX-317647630 | 22 | 5379664 | 1 | A/G | 5 |
| AX-317647663 | 22 | 5400755 | 0.961494 | C/T | 5 |
| AX-317647753 | 22 | 5479097 | 0.888508 | A/C | 5 |
| AX-317647967 | 22 | 5624384 | 0.801924 | G/T | 5 |
| AX-317647975 | 22 | 5629013 | 0.868268 | T/G | 5 |
| AX-317618916 | 22 | 5643839 | 0.830893 | G/A | 5 |
| AX-317648158 | 22 | 5759430 | 0.87552 | C/T | 5 |
| AX-317619074 | 22 | 5785936 | 0.836102 | A/G | 5 |
| AX-317648270 | 22 | 5898003 | 0.891089 | A/G | 5 |
| AX-317619434 | 22 | 6046709 | 0.771968 | A/G | 5 |
| AX-317648645 | 22 | 6115097 | 0.722038 | G/A | 5 |
| AX-317648814 | 22 | 6357134 | 0.745915 | C/T | 5 |
| AX-323025320 | 22 | 6421645 | 0.772431 | C/T | 5 |
| AX-317619837 | 22 | 6467900 | 0.771968 | G/A | 5 |
| AX-317649322 | 22 | 8238228 | 0.768192 | A/G | 6 |
| AX-317649351 | 22 | 8247697 | 0.661803 | A/G | 6 |
| AX-317620240 | 22 | 8270779 | 0.770525 | A/C | 6 |
| AX-317620303 | 22 | 8780810 | 0.654261 | A/G | 6 |
| AX-317620317 | 22 | 8916395 | 0.772431 | T/C | 6 |
| AX-317620336 | 22 | 8934022 | 0.672979 | A/G | 6 |
| AX-317649462 | 22 | 8936400 | 0.65763 | G/A | 6 |
| AX-317620358 | 22 | 8965147 | 0.772431 | G/A | 6 |
| AX-317649538 | 22 | 9036873 | 0.768192 | A/G | 6 |
| AX-317620474 | 22 | 9097151 | 0.716172 | A/C | 6 |
| AX-317649879 | 22 | 9236300 | 0.734218 | C/T | 6 |
| AX-317649969 | 22 | 9299308 | 0.637702 | G/A | 6 |
| AX-317620863 | 22 | 9336674 | 0.658065 | T/C | 6 |
| AX-317035348 | 22 | 9438257 | 0.632511 | A/G | 6 |
| AX-317650234 | 22 | 9508455 | 0.623891 | A/C | 6 |
| AX-317621349 | 22 | 9718148 | 1 | A/G | 6 |
| AX-317621446 | 22 | 9769434 | 0.810046 | G/A | 6 |
| AX-317650800 | 22 | 9864042 | 0.976302 | A/C | 6 |
| AX-317621808 | 22 | 9969880 | 0.861518 | G/A | 6 |

TABLE 6
Candidate genes flanking the remaining significant SNPs

| | QTL region^{a} | | |
|---|---|---|---|
| | Left | Right | |
| SNP | position | Position | Genes Names |
| AX-317619074 | 5285936 | 6285936 | cacng7, cacng6, cacng4, vamp2, styk1, glut1, phc1, hlf, |
| | | | grind2d, iffo1, gapdh, gtan, cox6b1, chd4, clcn1, cdc42, clec6a |
| AX-317617531 | 1388302 | 2388302 | zbed1, zscan2, tmem65, sema6d, tatdn1, pomc, nadh, mtss1, |
| | | | grik5, fibcd1, rnf139, ceacam5 |
| AX-317616778 | 1 | 776593 | zhx1, vps52, senp1, muc5ac, ha1f, rnf2, atad2, rps18 |
| AX-317648645 | 5615097 | 6615097 | cacng7, cacng6, cacng4, tmem238, glut1, sbk2, rcvrn, shisa7, phc1, |
| | | | hlf, grind2d/ccdc106, il11, gsg1l, gtan, fpr2, fpr1, epn1, cox6b1, |
| | | | cnot2, c3ar1, clec6a, necap1 |
| AX-317617852 | 1932164 | 2932164 | trnal-aag, zbed1, pde, tnfrsf6b, tmem65, scgn, tatdn1, pomc, mtss1, |
| | | | grik5, fibcd1, carmil1, enpp1/enpp2, rnf139, deptor, col14a1, |
| | | | cd209, atx2 |
| AX-317616808 | 260642 | 1260642 | zhx2, zhx1, psbp2, af1q, psmb4, plekho1, ha1f, gabpb2, rfx5, |
| | | | cdc42se1, clec12a, bcl2/bnip3l, atad2, anp32e |
| AX-317647368 | 2978052 | 3978052 | tnc, harbi1, muc1, mr1, atf6b, lhx9, h2q10, h2d1, hua2, cd209e, |
| | | | cd209d, cd209c, cd20912, clec4e |
| AX-317647975 | 5129013 | 6129013 | ivl cacng7, cacng6, cacng4, vamp2, styk1, trim29, tpi1, glut1, phc1, |
| | | | grin2d, iffo2, gnb3, gapdh, gtan, eno2, trim21, cox6b1, chd4, clcn1, |
| | | | cdc42, cd209e, clec6a |
| AX-317621349 | 9218148 | 10218148 | znf706, atp1a3, clc, pou2f2, nectin2, NADH, macf1, asgr1, gsk3b, erf, |
| | | | rnf19b, cd209e, cd209a, bmp8a, ak2 |
| AX-317617390 | 1258060 | 2258060 | zbed1, zscan2, unc tbtbp, mindy1, sema6d, pomc, pi4kb, grik5, rfx5, |
| | | | ceacam5 |
| AX-317647016 | 2021284 | 3021284 | trnal-aag, zbed1, pde, tnfrsf6b, tmem65, scgn, tatdn1, pomc, mtss1, |
| | | | grik5, fibcd1, carmil1, enpp1/enpp2, rnf139, deptor, col14a1, cd209, |
| | | | atx2 |
| AX-317618485 | 4789967 | 5789967 | cacng7, cacng6, cacng4, vamp2, styk1, glut1, phc1, hlf, grind2d, |
| | | | iffo1, gapdh, gtan, cox6b1, chd4, clcn1, cdc42, clec6a, cd209e |
| AX-317649969 | 8799308 | 9799308 | trnag-ccc, trnag-ccc, trnag-ccc, znf706, znf384, zbtb22, tubb, tcf19, |
| | | | syncytin2, atp1a3, cic, pou2f2, nectin2, macf1, gsk3b, flot1, erf, |
| | | | bmp8a |
| AX-317647630 | 4879664 | 5879664 | cdc42, chd4, vamp2, iffo1, gapdh, ivl |
| AX-317620863 | 8836674 | 9836674 | trnag-ccc, trnag-ccc, trnag-ccc, znf706, znf384, zbtb22, tubb, tcf19, |
| | | | syncytin2, atp1a3, cic, pou2f2, nectin2, macf1, gsk3b, flot1, erf, |
| | | | bmp8a |
| AX-317647753 | 4979097 | 5979097 | cacgn7, cacgn6, cacgn4, vamp2, styk1, trim29, tpi1, glut1, phc1, |
| | | | iffo2, gnb3, gapdh, gtan, eno2, g2e3, trim21, cox6b1, chd4, clcn1, |
| | | | cdc42, cd209e, clec6a |
| AX-317617254 | 1116487 | 2116487 | zscan2, mindy1, sema6d, af1q, psmb4, plekha6, pi4kb, grik5, |
| | | | gabpb2, rfx5, cdc42se1, ceacam5, bcl2/bnip2, fp1, anp32a |
| AX-317646938 | 1945275 | 2945275 | trnal-aag, zbed1, pde, tnfrsf6b, tmem65, scgn, tatdn1, pomc, mtss1, |
| | | | grik5, fibcd1, carmil1, enpp1/enpp2, rnf139, deptor, col14a1, cd209, |
| | | | atx2 |
| AX-317646411 | 1214252 | 2214252 | zbed1, zscan2, unc tbtbp, mindy1, sema6d, pomc, pi4kb, grik5, rfx5, |
| | | | ceacam5, bcl2/bnip2, fp1 |
| AX-317617977 | 2055673 | 3055673 | trnal-aag, zbed1, pde, tnfrsf6b, tmem65, scgn, tatdn1, pomc, mtss1, |
| | | | grik5, fibcd1, carmil1, enpp1/enpp2, rnf139, deptor, col14a1, cd209, |
| | | | atx2 |
| AX-317620317 | 8416395 | 9416395 | trnag-ccc, trnag-ccc, trnag-ccc, znf384, zbtb22, tubb, tcf19, tap, |
| | | | syncytin2, nectin2, mr1, h2q9, zg49, g2e3, flot1, daxx, ha1f, kifc3 |
| AX-317063470 | 941242 | 1941242 | zhx2, zscan2, mindy1, sema6d, psbp2, af1q, psmb4, plekha6, pi4kb, |
| | | | grik5, gabpb22, rfx5, cdc42se1, ceacam5, clec12a/bcl2/bnip2, fp2, |
| | | | anp32a |
| AX-317646673 | 1488041 | 2488041 | trnal-aag, zbed1, zscan2, tnfrsf6b, tmem65, sema6d, scgn, tatdn1, |
| | | | pomc, NADH, mtss1, grik5, fibcd1, carmil1, rnf139, ceacam5, atx2 |
| AX-317617288 | 1160924 | 2160924 | zbed1, zscan2, unc tbtbp, mindy1, sema6d, af1q, psmb4, pi4kb, |
| | | | grik5, gabpb2, rfx5, cdc42se1, ceacam5, bcl2/bnip2, fp1, anp32a |
| AX-317648270 | 5398003 | 6398003 | cacng7, cacng6, cacng4, vamp2, styk1, glut1, shisa7, phc1, hlf, |
| | | | grin2d, iffo1, gapdh, gtan, coxb61, chd4, clcn1, cnot3, clec6a |

^{a}QTL region size was defined as 500 kb upstream and downstream the position of the SNP.

TABLE 7
Significant SNPs associated with TiLV defined as BS identified
through a fine-mapping whole genome sequencing data

| SNP # | SNP Id | BP | P |
|---|---|---|---|
| 1 | 22:311907 | 311907 | 4.70E−11 |
| 2 | 22:315033 | 315033 | 4.70E−11 |
| 3 | 22:315200 | 315200 | 4.70E−11 |
| 4 | 22:1945397 | 1945397 | 5.39E−11 |
| 5 | 22:323396 | 323396 | 5.92E−11 |
| 6 | 22:316028 | 316028 | 7.53E−11 |
| 7 | 22:316516 | 316516 | 7.53E−11 |
| 8 | 22:319781 | 319781 | 7.53E−11 |
| 9 | 22:323389 | 323389 | 7.53E−11 |
| 10 | 22:324000 | 324000 | 7.53E−11 |
| 11 | 22:340118 | 340118 | 7.53E−11 |
| 12 | 22:1937264 | 1937264 | 8.20E−11 |
| 13 | 22:240620 | 240620 | 8.80E−11 |
| 14 | 22:312755 | 312755 | 9.20E−11 |
| 15 | 22:312757 | 312757 | 9.20E−11 |
| 16 | 22:1928074 | 1928074 | 9.75E−11 |
| 17 | 22:140555 | 140555 | 1.05E−10 |
| 18 | 22:340795 | 340795 | 1.13E−10 |
| 19 | 22:1927702 | 1927702 | 1.27E−10 |
| 20 | 22:5191861 | 5191861 | 1.29E−10 |
| 21 | 22:320074 | 320074 | 1.31E−10 |
| 22 | 22:142955 | 142955 | 1.41E−10 |
| 23 | 22:333568 | 333568 | 1.44E−10 |
| 24 | 22:309419 | 309419 | 1.63E−10 |
| 25 | 22:323203 | 323203 | 1.83E−10 |
| 26 | 22:323208 | 323208 | 1.83E−10 |
| 27 | 22:5276725 | 5276725 | 1.90E−10 |
| 28 | 22:8226272 | 8226272 | 1.93E−10 |
| 29 | 22:310900 | 310900 | 1.98E−10 |
| 30 | 22:9116842 | 9116842 | 2.34E−10 |
| 31 | 22:1941767 | 1941767 | 2.60E−10 |
| 32 | 22:307805 | 307805 | 2.69E−10 |
| 33 | 22:307817 | 307817 | 2.69E−10 |
| 34 | 22:349313 | 349313 | 2.73E−10 |
| 35 | 22:243425 | 243425 | 2.74E−10 |
| 36 | 22:249500 | 249500 | 2.74E−10 |
| 37 | 22:233193 | 233193 | 2.89E−10 |
| 38 | 22:9300046 | 9300046 | 2.95E−10 |
| 39 | 22:8225599 | 8225599 | 2.95E−10 |
| 40 | 22:310297 | 310297 | 3.03E−10 |
| 41 | 22:8225465 | 8225465 | 3.45E−10 |
| 42 | 22:1909889 | 1909889 | 3.52E−10 |
| 43 | 22:255104 | 255104 | 3.58E−10 |
| 44 | 22:251435 | 251435 | 3.85E−10 |
| 45 | 22:251476 | 251476 | 3.85E−10 |
| 46 | 22:251481 | 251481 | 3.85E−10 |
| 47 | 22:251570 | 251570 | 3.85E−10 |
| 48 | 22:251578 | 251578 | 3.85E−10 |
| 49 | 22:251579 | 251579 | 3.85E−10 |
| 50 | 22:251629 | 251629 | 3.85E−10 |
| 51 | 22:251748 | 251748 | 3.85E−10 |
| 52 | 22:251794 | 251794 | 3.85E−10 |
| 53 | 22:251811 | 251811 | 3.85E−10 |
| 54 | 22:251812 | 251812 | 3.85E−10 |
| 55 | 22:252267 | 252267 | 3.85E−10 |
| 56 | 22:252348 | 252348 | 3.85E−10 |
| 57 | 22:252389 | 252389 | 3.85E−10 |
| 58 | 22:252682 | 252682 | 3.85E−10 |
| 59 | 22:253414 | 253414 | 3.85E−10 |
| 60 | 22:253516 | 253516 | 3.85E−10 |
| 61 | 22:253589 | 253589 | 3.85E−10 |
| 62 | 22:254378 | 254378 | 3.85E−10 |
| 63 | 22:254383 | 254383 | 3.85E−10 |
| 64 | 22:255019 | 255019 | 3.85E−10 |
| 65 | 22:255038 | 255038 | 3.85E−10 |
| 66 | 22:255458 | 255458 | 3.85E−10 |
| 67 | 22:256474 | 256474 | 3.85E−10 |
| 68 | 22:256561 | 256561 | 3.85E−10 |
| 69 | 22:257266 | 257266 | 3.85E−10 |
| 70 | 22:257674 | 257674 | 3.85E−10 |
| 71 | 22:257711 | 257711 | 3.85E−10 |
| 72 | 22:257716 | 257716 | 3.85E−10 |
| 73 | 22:257733 | 257733 | 3.85E−10 |
| 74 | 22:259439 | 259439 | 3.85E−10 |
| 75 | 22:259543 | 259543 | 3.85E−10 |
| 76 | 22:259567 | 259567 | 3.85E−10 |
| 77 | 22:259622 | 259622 | 3.85E−10 |
| 78 | 22:259627 | 259627 | 3.85E−10 |
| 79 | 22:259659 | 259659 | 3.85E−10 |
| 80 | 22:259844 | 259844 | 3.85E−10 |
| 81 | 22:259880 | 259880 | 3.85E−10 |
| 82 | 22:259900 | 259900 | 3.85E−10 |
| 83 | 22:312605 | 312605 | 3.85E−10 |
| 84 | 22:312689 | 312689 | 3.85E−10 |
| 85 | 22:312691 | 312691 | 3.85E−10 |
| 86 | 22:312856 | 312856 | 3.85E−10 |
| 87 | 22:313509 | 313509 | 3.85E−10 |
| 88 | 22:313584 | 313584 | 3.85E−10 |
| 89 | 22:314751 | 314751 | 3.85E−10 |
| 90 | 22:314948 | 314948 | 3.85E−10 |
| 91 | 22:315231 | 315231 | 3.85E−10 |
| 92 | 22:315722 | 315722 | 3.85E−10 |
| 93 | 22:315830 | 315830 | 3.85E−10 |
| 94 | 22:316880 | 316880 | 3.85E−10 |
| 95 | 22:318187 | 318187 | 3.85E−10 |
| 96 | 22:319672 | 319672 | 3.85E−10 |
| 97 | 22:319704 | 319704 | 3.85E−10 |
| 98 | 22:319872 | 319872 | 3.85E−10 |
| 99 | 22:319938 | 319938 | 3.85E−10 |
| 100 | 22:322562 | 322562 | 3.85E−10 |
| 101 | 22:322571 | 322571 | 3.85E−10 |
| 102 | 22:323172 | 323172 | 3.85E−10 |
| 103 | 22:323269 | 323269 | 3.85E−10 |
| 104 | 22:323320 | 323320 | 3.85E−10 |
| 105 | 22:323487 | 323487 | 3.85E−10 |
| 106 | 22:324925 | 324925 | 3.85E−10 |
| 107 | 22:325121 | 325121 | 3.85E−10 |
| 108 | 22:325710 | 325710 | 3.85E−10 |
| 109 | 22:329868 | 329868 | 3.85E−10 |
| 110 | 22:330018 | 330018 | 3.85E−10 |
| 111 | 22:332020 | 332020 | 3.85E−10 |
| 112 | 22:332525 | 332525 | 3.85E−10 |
| 113 | 22:332655 | 332655 | 3.85E−10 |
| 114 | 22:332687 | 332687 | 3.85E−10 |
| 115 | 22:332962 | 332962 | 3.85E−10 |
| 116 | 22:333252 | 333252 | 3.85E−10 |
| 117 | 22:333392 | 333392 | 3.85E−10 |
| 118 | 22:335082 | 335082 | 3.85E−10 |
| 119 | 22:335480 | 335480 | 3.85E−10 |
| 120 | 22:336053 | 336053 | 3.85E−10 |
| 121 | 22:336532 | 336532 | 3.85E−10 |
| 122 | 22:336902 | 336902 | 3.85E−10 |
| 123 | 22:336903 | 336903 | 3.85E−10 |
| 124 | 22:337122 | 337122 | 3.85E−10 |
| 125 | 22:337729 | 337729 | 3.85E−10 |
| 126 | 22:1929638 | 1929638 | 4.11E−10 |
| 127 | 22:1953523 | 1953523 | 4.30E−10 |
| 128 | 22:142958 | 142958 | 4.89E−10 |
| 129 | 22:260163 | 260163 | 5.03E−10 |
| 130 | 22:260374 | 260374 | 5.03E−10 |
| 131 | 22:260375 | 260375 | 5.03E−10 |
| 132 | 22:260450 | 260450 | 5.03E−10 |
| 133 | 22:260528 | 260528 | 5.03E−10 |
| 134 | 22:260982 | 260982 | 5.03E−10 |
| 135 | 22:261065 | 261065 | 5.03E−10 |
| 136 | 22:264873 | 264873 | 5.03E−10 |
| 137 | 22:264947 | 264947 | 5.03E−10 |
| 138 | 22:264979 | 264979 | 5.03E−10 |
| 139 | 22:265019 | 265019 | 5.03E−10 |
| 140 | 22:265022 | 265022 | 5.03E−10 |
| 141 | 22:265032 | 265032 | 5.03E−10 |
| 142 | 22:265052 | 265052 | 5.03E−10 |
| 143 | 22:265113 | 265113 | 5.03E−10 |
| 144 | 22:265127 | 265127 | 5.03E−10 |
| 145 | 22:265194 | 265194 | 5.03E−10 |
| 146 | 22:265455 | 265455 | 5.03E−10 |
| 147 | 22:265519 | 265519 | 5.03E−10 |
| 148 | 22:265642 | 265642 | 5.03E−10 |
| 149 | 22:265643 | 265643 | 5.03E−10 |
| 150 | 22:265721 | 265721 | 5.03E−10 |
| 151 | 22:266071 | 266071 | 5.03E−10 |
| 152 | 22:266212 | 266212 | 5.03E−10 |
| 153 | 22:266280 | 266280 | 5.03E−10 |
| 154 | 22:266345 | 266345 | 5.03E−10 |
| 155 | 22:266726 | 266726 | 5.03E−10 |
| 156 | 22:267367 | 267367 | 5.03E−10 |
| 157 | 22:267416 | 267416 | 5.03E−10 |
| 158 | 22:267428 | 267428 | 5.03E−10 |
| 159 | 22:267465 | 267465 | 5.03E−10 |
| 160 | 22:267991 | 267991 | 5.03E−10 |
| 161 | 22:267994 | 267994 | 5.03E−10 |
| 162 | 22:9248284 | 9248284 | 5.11E−10 |
| 163 | 22:9255499 | 9255499 | 5.11E−10 |
| 164 | 22:9255639 | 9255639 | 5.11E−10 |
| 165 | 22:9256431 | 9256431 | 5.11E−10 |
| 166 | 22:8268672 | 8268672 | 5.15E−10 |
| 167 | 22:2451403 | 2451403 | 5.15E−10 |
| 168 | 22:1939192 | 1939192 | 5.39E−10 |
| 169 | 22:348288 | 348288 | 5.48E−10 |
| 170 | 22:8261780 | 8261780 | 5.49E−10 |
| 171 | 22:8261869 | 8261869 | 5.49E−10 |
| 172 | 22:318188 | 318188 | 5.57E−10 |
| 173 | 22:321693 | 321693 | 5.57E−10 |
| 174 | 22:336735 | 336735 | 5.57E−10 |
| 175 | 22:339372 | 339372 | 5.57E−10 |
| 176 | 22:340616 | 340616 | 5.75E−10 |
| 177 | 22:340737 | 340737 | 5.75E−10 |
| 178 | 22:340891 | 340891 | 5.75E−10 |
| 179 | 22:340906 | 340906 | 5.75E−10 |
| 180 | 22:340989 | 340989 | 5.75E−10 |
| 181 | 22:341895 | 341895 | 5.75E−10 |
| 182 | 22:8227643 | 8227643 | 5.78E−10 |
| 183 | 22:9148797 | 9148797 | 5.85E−10 |
| 184 | 22:1929846 | 1929846 | 6.00E−10 |
| 185 | 22:9247218 | 9247218 | 6.43E−10 |
| 186 | 22:8240537 | 8240537 | 6.47E−10 |
| 187 | 22:268171 | 268171 | 6.49E−10 |
| 188 | 22:268197 | 268197 | 6.49E−10 |
| 189 | 22:268239 | 268239 | 6.49E−10 |
| 190 | 22:268305 | 268305 | 6.49E−10 |
| 191 | 22:268306 | 268306 | 6.49E−10 |
| 192 | 22:268345 | 268345 | 6.49E−10 |
| 193 | 22:268440 | 268440 | 6.49E−10 |
| 194 | 22:268480 | 268480 | 6.49E−10 |
| 195 | 22:268482 | 268482 | 6.49E−10 |
| 196 | 22:269929 | 269929 | 6.49E−10 |
| 197 | 22:269938 | 269938 | 6.49E−10 |
| 198 | 22:270126 | 270126 | 6.49E−10 |
| 199 | 22:270135 | 270135 | 6.49E−10 |
| 200 | 22:270146 | 270146 | 6.49E−10 |
| 201 | 22:270300 | 270300 | 6.49E−10 |
| 202 | 22:270370 | 270370 | 6.49E−10 |
| 203 | 22:270446 | 270446 | 6.49E−10 |
| 204 | 22:271053 | 271053 | 6.49E−10 |
| 205 | 22:271939 | 271939 | 6.49E−10 |
| 206 | 22:271943 | 271943 | 6.49E−10 |
| 207 | 22:271947 | 271947 | 6.49E−10 |
| 208 | 22:272833 | 272833 | 6.49E−10 |
| 209 | 22:273079 | 273079 | 6.49E−10 |
| 210 | 22:275097 | 275097 | 6.49E−10 |
| 211 | 22:276511 | 276511 | 6.49E−10 |
| 212 | 22:276788 | 276788 | 6.49E−10 |
| 213 | 22:278768 | 278768 | 6.49E−10 |
| 214 | 22:278786 | 278786 | 6.49E−10 |
| 215 | 22:279021 | 279021 | 6.49E−10 |
| 216 | 22:279145 | 279145 | 6.49E−10 |
| 217 | 22:281281 | 281281 | 6.49E−10 |
| 218 | 22:281316 | 281316 | 6.49E−10 |
| 219 | 22:281336 | 281336 | 6.49E−10 |
| 220 | 22:285781 | 285781 | 6.49E−10 |
| 221 | 22:285800 | 285800 | 6.49E−10 |
| 222 | 22:285811 | 285811 | 6.49E−10 |
| 223 | 22:285817 | 285817 | 6.49E−10 |
| 224 | 22:285818 | 285818 | 6.49E−10 |
| 225 | 22:286065 | 286065 | 6.49E−10 |
| 226 | 22:287376 | 287376 | 6.49E−10 |
| 227 | 22:287394 | 287394 | 6.49E−10 |
| 228 | 22:287399 | 287399 | 6.49E−10 |
| 229 | 22:293118 | 293118 | 6.49E−10 |
| 230 | 22:293127 | 293127 | 6.49E−10 |
| 231 | 22:293427 | 293427 | 6.49E−10 |
| 232 | 22:304414 | 304414 | 6.49E−10 |
| 233 | 22:304440 | 304440 | 6.49E−10 |
| 234 | 22:304447 | 304447 | 6.49E−10 |
| 235 | 22:304460 | 304460 | 6.49E−10 |
| 236 | 22:304542 | 304542 | 6.49E−10 |
| 237 | 22:305246 | 305246 | 6.49E−10 |
| 238 | 22:305667 | 305667 | 6.49E−10 |
| 239 | 22:305732 | 305732 | 6.49E−10 |
| 240 | 22:307493 | 307493 | 6.49E−10 |
| 241 | 22:312756 | 312756 | 6.56E−10 |
| 242 | 22:320067 | 320067 | 6.62E−10 |
| 243 | 22:1943830 | 1943830 | 6.80E−10 |
| 244 | 22:9215645 | 9215645 | 6.96E−10 |
| 245 | 22:2575622 | 2575622 | 7.20E−10 |
| 246 | 22:321595 | 321595 | 7.24E−10 |
| 247 | 22:337895 | 337895 | 7.24E−10 |
| 248 | 22:339470 | 339470 | 7.24E−10 |
| 249 | 22:9112492 | 9112492 | 7.34E−10 |
| 250 | 22:9073996 | 9073996 | 7.48E−10 |
| 251 | 22:8225346 | 8225346 | 7.78E−10 |
| 252 | 22:8225350 | 8225350 | 7.78E−10 |
| 253 | 22:8373431 | 8373431 | 7.98E−10 |
| 254 | 22:222611 | 222611 | 8.00E−10 |
| 255 | 22:5541093 | 5541093 | 8.27E−10 |
| 256 | 22:5541614 | 5541614 | 8.27E−10 |
| 257 | 22:5208023 | 5208023 | 8.68E−10 |
| 258 | 22:218845 | 218845 | 9.15E−10 |
| 259 | 22:1337914 | 1337914 | 9.82E−10 |
| 260 | 22:9149942 | 9149942 | 1.08E−09 |
| 261 | 22:2493176 | 2493176 | 1.08E−09 |
| 262 | 22:353919 | 353919 | 1.10E−09 |
| 263 | 22:9157250 | 9157250 | 1.16E−09 |
| 264 | 22:9158346 | 9158346 | 1.16E−09 |
| 265 | 22:9160702 | 9160702 | 1.16E−09 |
| 266 | 22:5647544 | 5647544 | 1.21E−09 |
| 267 | 22:310671 | 310671 | 1.22E−09 |
| 268 | 22:187999 | 187999 | 1.25E−09 |
| 269 | 22:189632 | 189632 | 1.25E−09 |
| 270 | 22:203690 | 203690 | 1.25E−09 |
| 271 | 22:8275134 | 8275134 | 1.25E−09 |
| 272 | 22:5542517 | 5542517 | 1.27E−09 |
| 273 | 22:9192276 | 9192276 | 1.27E−09 |
| 274 | 22:1922859 | 1922859 | 1.29E−09 |
| 275 | 22:1926052 | 1926052 | 1.29E−09 |
| 276 | 22:2576678 | 2576678 | 1.31E−09 |
| 277 | 22:328358 | 328358 | 1.34E−09 |
| 278 | 22:328447 | 328447 | 1.34E−09 |
| 279 | 22:328593 | 328593 | 1.34E−09 |
| 280 | 22:329165 | 329165 | 1.34E−09 |
| 281 | 22:8292185 | 8292185 | 1.35E−09 |
| 282 | 22:6587159 | 6587159 | 1.36E−09 |
| 283 | 22:2539310 | 2539310 | 1.37E−09 |
| 284 | 22:2539659 | 2539659 | 1.37E−09 |
| 285 | 22:9248287 | 9248287 | 1.47E−09 |
| 286 | 22:9202733 | 9202733 | 1.52E−09 |
| 287 | 22:9037380 | 9037380 | 1.59E−09 |
| 288 | 22:8262024 | 8262024 | 1.59E−09 |
| 289 | 22:1970461 | 1970461 | 1.59E−09 |
| 290 | 22:8254320 | 8254320 | 1.61E−09 |
| 291 | 22:8360141 | 8360141 | 1.65E−09 |
| 292 | 22:318117 | 318117 | 1.70E−09 |
| 293 | 22:187185 | 187185 | 1.71E−09 |
| 294 | 22:8909521 | 8909521 | 1.74E−09 |
| 295 | 22:329322 | 329322 | 1.79E−09 |
| 296 | 22:8248839 | 8248839 | 1.84E−09 |
| 297 | 22:308924 | 308924 | 1.86E−09 |
| 298 | 22:6571355 | 6571355 | 1.90E−09 |
| 299 | 22:8267340 | 8267340 | 1.90E−09 |
| 300 | 22:8268105 | 8268105 | 1.90E−09 |
| 301 | 22:8268675 | 8268675 | 1.90E−09 |
| 302 | 22:8268964 | 8268964 | 1.90E−09 |
| 303 | 22:8269955 | 8269955 | 1.90E−09 |
| 304 | 22:8271755 | 8271755 | 1.90E−09 |
| 305 | 22:8272029 | 8272029 | 1.90E−09 |
| 306 | 22:8273555 | 8273555 | 1.90E−09 |
| 307 | 22:308057 | 308057 | 1.91E−09 |
| 308 | 22:145679 | 145679 | 1.92E−09 |
| 309 | 22:2730251 | 2730251 | 1.93E−09 |
| 310 | 22:2750445 | 2750445 | 1.93E−09 |
| 311 | 22:310057 | 310057 | 1.96E−09 |
| 312 | 22:2493183 | 2493183 | 1.97E−09 |
| 313 | 22:1961350 | 1961350 | 2.07E−09 |
| 314 | 22:9221692 | 9221692 | 2.13E−09 |
| 315 | 22:9224613 | 9224613 | 2.13E−09 |
| 316 | 22:5294335 | 5294335 | 2.15E−09 |
| 317 | 22:5294347 | 5294347 | 2.15E−09 |
| 318 | 22:1961198 | 1961198 | 2.17E−09 |
| 319 | 22:307761 | 307761 | 2.17E−09 |
| 320 | 22:307991 | 307991 | 2.17E−09 |
| 321 | 22:308304 | 308304 | 2.17E−09 |
| 322 | 22:308307 | 308307 | 2.17E−09 |
| 323 | 22:1760998 | 1760998 | 2.22E−09 |
| 324 | 22:9010838 | 9010838 | 2.27E−09 |
| 325 | 22:5643994 | 5643994 | 2.29E−09 |
| 326 | 22:8262599 | 8262599 | 2.34E−09 |
| 327 | 22:3515083 | 3515083 | 2.35E−09 |
| 328 | 22:3515907 | 3515907 | 2.35E−09 |
| 329 | 22:143793 | 143793 | 2.47E−09 |
| 330 | 22:144207 | 144207 | 2.49E−09 |
| 331 | 22:6471008 | 6471008 | 2.52E−09 |
| 332 | 22:5603994 | 5603994 | 2.55E−09 |
| 333 | 22:260087 | 260087 | 2.58E−09 |
| 334 | 22:1816890 | 1816890 | 2.60E−09 |
| 335 | 22:9051521 | 9051521 | 2.64E−09 |
| 336 | 22:6459860 | 6459860 | 2.65E−09 |
| 337 | 22:2717487 | 2717487 | 2.67E−09 |
| 338 | 22:267335 | 267335 | 2.71E−09 |
| 339 | 22:5683460 | 5683460 | 2.75E−09 |
| 340 | 22:5683471 | 5683471 | 2.75E−09 |
| 341 | 22:1983947 | 1983947 | 2.81E−09 |
| 342 | 22:239073 | 239073 | 2.82E−09 |
| 343 | 22:308064 | 308064 | 2.84E−09 |
| 344 | 22:354572 | 354572 | 2.85E−09 |
| 345 | 22:8263244 | 8263244 | 2.91E−09 |
| 346 | 22:8960489 | 8960489 | 2.93E−09 |
| 347 | 22:8261357 | 8261357 | 2.94E−09 |
| 348 | 22:2477324 | 2477324 | 3.00E−09 |
| 349 | 22:8298781 | 8298781 | 3.02E−09 |
| 350 | 22:8298797 | 8298797 | 3.02E−09 |
| 351 | 22:8393882 | 8393882 | 3.05E−09 |
| 352 | 22:185723 | 185723 | 3.06E−09 |
| 353 | 22:8359021 | 8359021 | 3.07E−09 |
| 354 | 22:8393116 | 8393116 | 3.08E−09 |
| 355 | 22:8333257 | 8333257 | 3.09E−09 |
| 356 | 22:348694 | 348694 | 3.10E−09 |
| 357 | 22:176901 | 176901 | 3.13E−09 |
| 358 | 22:348685 | 348685 | 3.15E−09 |
| 359 | 22:8393820 | 8393820 | 3.21E−09 |
| 360 | 22:9285749 | 9285749 | 3.22E−09 |
| 361 | 22:9285750 | 9285750 | 3.22E−09 |
| 362 | 22:9285894 | 9285894 | 3.22E−09 |
| 363 | 22:9286075 | 9286075 | 3.22E−09 |
| 364 | 22:9286493 | 9286493 | 3.22E−09 |
| 365 | 22:9300838 | 9300838 | 3.22E−09 |
| 366 | 22:8301138 | 8301138 | 3.23E−09 |
| 367 | 22:8241475 | 8241475 | 3.24E−09 |
| 368 | 22:8247291 | 8247291 | 3.24E−09 |
| 369 | 22:8248934 | 8248934 | 3.24E−09 |
| 370 | 22:8248952 | 8248952 | 3.24E−09 |
| 371 | 22:8249044 | 8249044 | 3.24E−09 |
| 372 | 22:260083 | 260083 | 3.29E−09 |
| 373 | 22:270549 | 270549 | 3.29E−09 |
| 374 | 22:276792 | 276792 | 3.29E−09 |
| 375 | 22:277914 | 277914 | 3.29E−09 |
| 376 | 22:277917 | 277917 | 3.29E−09 |
| 377 | 22:971640 | 971640 | 3.42E−09 |
| 378 | 22:211399 | 211399 | 3.46E−09 |
| 379 | 22:215856 | 215856 | 3.46E−09 |
| 380 | 22:218813 | 218813 | 3.46E−09 |
| 381 | 22:9247028 | 9247028 | 3.54E−09 |
| 382 | 22:9259559 | 9259559 | 3.54E−09 |
| 383 | 22:9259576 | 9259576 | 3.54E−09 |
| 384 | 22:9259578 | 9259578 | 3.54E−09 |
| 385 | 22:9259645 | 9259645 | 3.54E−09 |
| 386 | 22:5603028 | 5603028 | 3.59E−09 |
| 387 | 22:5652482 | 5652482 | 3.61E−09 |
| 388 | 22:5653827 | 5653827 | 3.61E−09 |
| 389 | 22:215667 | 215667 | 3.62E−09 |
| 390 | 22:8258850 | 8258850 | 3.71E−09 |
| 391 | 22:8276518 | 8276518 | 3.72E−09 |
| 392 | 22:312803 | 312803 | 3.73E−09 |
| 393 | 22:9138074 | 9138074 | 3.77E−09 |
| 394 | 22:337897 | 337897 | 3.79E−09 |
| 395 | 22:9145233 | 9145233 | 3.79E−09 |
| 396 | 22:2474330 | 2474330 | 3.80E−09 |
| 397 | 22:5634421 | 5634421 | 3.81E−09 |
| 398 | 22:270116 | 270116 | 3.90E−09 |
| 399 | 22:293924 | 293924 | 3.90E−09 |
| 400 | 22:293960 | 293960 | 3.90E−09 |
| 401 | 22:294086 | 294086 | 3.90E−09 |
| 402 | 22:294788 | 294788 | 3.90E−09 |
| 403 | 22:9195387 | 9195387 | 3.98E−09 |
| 404 | 22:9195443 | 9195443 | 3.98E−09 |
| 405 | 22:9195809 | 9195809 | 3.98E−09 |
| 406 | 22:9195838 | 9195838 | 3.98E−09 |
| 407 | 22:9195871 | 9195871 | 3.98E−09 |
| 408 | 22:9196091 | 9196091 | 3.98E−09 |
| 409 | 22:9196150 | 9196150 | 3.98E−09 |
| 410 | 22:9196247 | 9196247 | 3.98E−09 |
| 411 | 22:9196668 | 9196668 | 3.98E−09 |
| 412 | 22:9196830 | 9196830 | 3.98E−09 |
| 413 | 22:9197017 | 9197017 | 3.98E−09 |
| 414 | 22:9197042 | 9197042 | 3.98E−09 |
| 415 | 22:5347447 | 5347447 | 4.03E−09 |
| 416 | 22:5348910 | 5348910 | 4.03E−09 |
| 417 | 22:2539889 | 2539889 | 4.12E−09 |
| 418 | 22:2541427 | 2541427 | 4.12E−09 |
| 419 | 22:2641786 | 2641786 | 4.13E−09 |
| 420 | 22:9104116 | 9104116 | 4.18E−09 |
| 421 | 22:144809 | 144809 | 4.22E−09 |
| 422 | 22:8300115 | 8300115 | 4.22E−09 |
| 423 | 22:8300622 | 8300622 | 4.22E−09 |
| 424 | 22:8302321 | 8302321 | 4.22E−09 |
| 425 | 22:213579 | 213579 | 4.22E−09 |
| 426 | 22:2718026 | 2718026 | 4.24E−09 |
| 427 | 22:2724512 | 2724512 | 4.27E−09 |
| 428 | 22:9275480 | 9275480 | 4.28E−09 |
| 429 | 22:5622447 | 5622447 | 4.29E−09 |
| 430 | 22:9176023 | 9176023 | 4.33E−09 |
| 431 | 22:9177988 | 9177988 | 4.36E−09 |
| 432 | 22:9179652 | 9179652 | 4.36E−09 |
| 433 | 22:9211403 | 9211403 | 4.36E−09 |
| 434 | 22:9214226 | 9214226 | 4.36E−09 |
| 435 | 22:8306628 | 8306628 | 4.40E−09 |
| 436 | 22:9148256 | 9148256 | 4.40E−09 |
| 437 | 22:9145858 | 9145858 | 4.55E−09 |
| 438 | 22:9165114 | 9165114 | 4.55E−09 |
| 439 | 22:8290232 | 8290232 | 4.62E−09 |
| 440 | 22:1939944 | 1939944 | 4.67E−09 |
| 441 | 22:1944161 | 1944161 | 4.67E−09 |
| 442 | 22:1945074 | 1945074 | 4.67E−09 |
| 443 | 22:9281654 | 9281654 | 4.70E−09 |
| 444 | 22:8394648 | 8394648 | 5.02E−09 |
| 445 | 22:8394649 | 8394649 | 5.02E−09 |
| 446 | 22:8394857 | 8394857 | 5.02E−09 |
| 447 | 22:8394869 | 8394869 | 5.02E−09 |
| 448 | 22:8394871 | 8394871 | 5.02E−09 |
| 449 | 22:8394926 | 8394926 | 5.02E−09 |
| 450 | 22:8396773 | 8396773 | 5.02E−09 |
| 451 | 22:8396806 | 8396806 | 5.02E−09 |
| 452 | 22:9331676 | 9331676 | 5.11E−09 |
| 453 | 22:5571107 | 5571107 | 5.15E−09 |
| 454 | 22:9368113 | 9368113 | 5.22E−09 |
| 455 | 22:9371177 | 9371177 | 5.22E−09 |
| 456 | 22:6456479 | 6456479 | 5.26E−09 |
| 457 | 22:6456505 | 6456505 | 5.26E−09 |
| 458 | 22:6456510 | 6456510 | 5.26E−09 |
| 459 | 22:9022088 | 9022088 | 5.28E−09 |
| 460 | 22:9024373 | 9024373 | 5.28E−09 |
| 461 | 22:9029574 | 9029574 | 5.28E−09 |
| 462 | 22:8262379 | 8262379 | 5.34E−09 |
| 463 | 22:9361594 | 9361594 | 5.45E−09 |
| 464 | 22:148261 | 148261 | 5.46E−09 |
| 465 | 22:149186 | 149186 | 5.46E−09 |
| 466 | 22:149753 | 149753 | 5.46E−09 |
| 467 | 22:8971144 | 8971144 | 5.47E−09 |
| 468 | 22:6604149 | 6604149 | 5.54E−09 |
| 469 | 22:6605124 | 6605124 | 5.54E−09 |
| 470 | 22:5597944 | 5597944 | 5.54E−09 |
| 471 | 22:6466646 | 6466646 | 5.56E−09 |
| 472 | 22:9300528 | 9300528 | 5.56E−09 |
| 473 | 22:307520 | 307520 | 5.58E−09 |
| 474 | 22:307575 | 307575 | 5.58E−09 |
| 475 | 22:2544895 | 2544895 | 5.68E−09 |
| 476 | 22:2760855 | 2760855 | 5.68E−09 |
| 477 | 22:2760857 | 2760857 | 5.68E−09 |
| 478 | 22:8974339 | 8974339 | 5.86E−09 |
| 479 | 22:172163 | 172163 | 5.89E−09 |
| 480 | 22:174494 | 174494 | 5.89E−09 |
| 481 | 22:175859 | 175859 | 5.89E−09 |
| 482 | 22:177725 | 177725 | 5.89E−09 |
| 483 | 22:189421 | 189421 | 5.89E−09 |
| 484 | 22:189441 | 189441 | 5.89E−09 |
| 485 | 22:189908 | 189908 | 5.89E−09 |
| 486 | 22:189922 | 189922 | 5.89E−09 |
| 487 | 22:192484 | 192484 | 5.89E−09 |
| 488 | 22:192950 | 192950 | 5.89E−09 |
| 489 | 22:200777 | 200777 | 5.89E−09 |
| 490 | 22:205122 | 205122 | 5.89E−09 |
| 491 | 22:205321 | 205321 | 5.89E−09 |
| 492 | 22:207862 | 207862 | 5.89E−09 |
| 493 | 22:207948 | 207948 | 5.89E−09 |
| 494 | 22:213870 | 213870 | 5.89E−09 |
| 495 | 22:5297813 | 5297813 | 5.89E−09 |
| 496 | 22:5298831 | 5298831 | 5.89E−09 |
| 497 | 22:9000538 | 9000538 | 5.89E−09 |
| 498 | 22:144755 | 144755 | 5.99E−09 |
| 499 | 22:5292935 | 5292935 | 6.04E−09 |
| 500 | 22:5595342 | 5595342 | 6.15E−09 |
| 501 | 22:3473756 | 3473756 | 6.15E−09 |
| 502 | 22:9054058 | 9054058 | 6.18E−09 |
| 503 | 22:5328800 | 5328800 | 6.40E−09 |
| 504 | 22:8945520 | 8945520 | 6.55E−09 |
| 505 | 22:8951545 | 8951545 | 6.55E−09 |
| 506 | 22:5345841 | 5345841 | 6.56E−09 |
| 507 | 22:5346210 | 5346210 | 6.56E−09 |
| 508 | 22:5584086 | 5584086 | 6.65E−09 |
| 509 | 22:8334048 | 8334048 | 6.66E−09 |
| 510 | 22:8360101 | 8360101 | 6.66E−09 |
| 511 | 22:5616161 | 5616161 | 6.67E−09 |
| 512 | 22:5361769 | 5361769 | 6.68E−09 |
| 513 | 22:8393072 | 8393072 | 6.73E−09 |
| 514 | 22:5991366 | 5991366 | 6.75E−09 |
| 515 | 22:142957 | 142957 | 6.87E−09 |
| 516 | 22:9009122 | 9009122 | 6.94E−09 |
| 517 | 22:5207914 | 5207914 | 7.10E−09 |
| 518 | 22:5207994 | 5207994 | 7.10E−09 |
| 519 | 22:9233322 | 9233322 | 7.19E−09 |
| 520 | 22:9235560 | 9235560 | 7.19E−09 |
| 521 | 22:8944704 | 8944704 | 7.33E−09 |
| 522 | 22:8990764 | 8990764 | 7.43E−09 |
| 523 | 22:9036723 | 9036723 | 7.46E−09 |
| 524 | 22:144748 | 144748 | 7.47E−09 |
| 525 | 22:1905662 | 1905662 | 7.50E−09 |
| 526 | 22:1780331 | 1780331 | 7.51E−09 |
| 527 | 22:1784171 | 1784171 | 7.51E−09 |
| 528 | 22:1786601 | 1786601 | 7.51E−09 |
| 529 | 22:1786775 | 1786775 | 7.51E−09 |
| 530 | 22:8929101 | 8929101 | 7.53E−09 |
| 531 | 22:1981160 | 1981160 | 7.65E−09 |
| 532 | 22:3605829 | 3605829 | 7.71E−09 |
| 533 | 22:3605843 | 3605843 | 7.71E−09 |
| 534 | 22:5598650 | 5598650 | 7.73E−09 |
| 535 | 22:8922632 | 8922632 | 7.78E−09 |
| 536 | 22:5219769 | 5219769 | 7.79E−09 |
| 537 | 22:5219831 | 5219831 | 7.79E−09 |
| 538 | 22:8903676 | 8903676 | 7.80E−09 |
| 539 | 22:8905856 | 8905856 | 7.80E−09 |
| 540 | 22:2542409 | 2542409 | 7.85E−09 |
| 541 | 22:142934 | 142934 | 7.89E−09 |
| 542 | 22:9227324 | 9227324 | 7.92E−09 |
| 543 | 22:5212093 | 5212093 | 8.08E−09 |
| 544 | 22:9320073 | 9320073 | 8.08E−09 |
| 545 | 22:5308405 | 5308405 | 8.17E−09 |
| 546 | 22:5308406 | 5308406 | 8.17E−09 |
| 547 | 22:5413154 | 5413154 | 8.26E−09 |
| 548 | 22:9284743 | 9284743 | 8.29E−09 |
| 549 | 22:9318681 | 9318681 | 8.29E−09 |
| 550 | 22:9318687 | 9318687 | 8.29E−09 |
| 551 | 22:9319105 | 9319105 | 8.29E−09 |
| 552 | 22:6455628 | 6455628 | 8.40E−09 |
| 553 | 22:6455740 | 6455740 | 8.40E−09 |
| 554 | 22:6456438 | 6456438 | 8.40E−09 |
| 555 | 22:1984321 | 1984321 | 8.43E−09 |
| 556 | 22:8976704 | 8976704 | 8.43E−09 |
| 557 | 22:8977310 | 8977310 | 8.43E−09 |
| 558 | 22:8977631 | 8977631 | 8.43E−09 |
| 559 | 22:8977633 | 8977633 | 8.43E−09 |
| 560 | 22:8978574 | 8978574 | 8.43E−09 |
| 561 | 22:8978591 | 8978591 | 8.43E−09 |
| 562 | 22:8283322 | 8283322 | 8.61E−09 |
| 563 | 22:9259582 | 9259582 | 8.65E−09 |
| 564 | 22:176846 | 176846 | 8.69E−09 |

1. A method of determining whether or not a tilapia may display increased resistance to infection by a virus, the method comprising genotyping the tilapia in order to identify one or more nucleotide alterations within chromosomes 22 and/or 3 and determining whether or not the tilapia is resistant, or likely to display increased resistance to infection by the virus, or likely to have offspring which display increased resistance to infection by the virus.

2. The method according to claim 1 wherein said one or more nucleotide alterations are found in a region of approximately 10 Mb, between nucleotides 1 and 10,000,000 on chromosome 22 and/or in a region of 2 Mb between nucleotides 70,700,000 and 72,700,000 on chromosome 3.

3. The method according to claim 1 wherein said one or more nucleotide alterations are found in a region of approximately 6.2 Mb, between 1 and 6,200,000 on chromosome 22.

4. The method according to 1 wherein said one or more nucleotide alterations are found in a region of approximately 2.0 Mb, between 1 and 2,000,000 on chromosome 22.

5. The method according to 1 wherein said one or more nucleotide alterations are found in a region of approximately 360 kb, between 1 and 360,000 on chromosome 22.

6. The method according to 1 wherein said one or more nucleotide alterations occurs on one or both copies of the identified chromosome.

7. The method according to 1 wherein said one or more nucleotide alterations comprises a substitution, deletion, inversion, addition, or duplication of one or more nucleotides.

8. The method according to 1 wherein said one or more nucleotide alterations comprises a SNP.

9. The method according to 1 wherein said one or more nucleotide alterations generates a nonsynonymous mutation in any of the genes mentioned in Table 3.

10. The method according to 8, wherein said SNP is one or more SNPS as identified in Table 2, or a SNP which is in linkage disequilibrium (LD) with one or more SNPs identified in Table 2.

11. The method according to claim 8 wherein the SNP which is in (LD) with one or more SNPs identified in Table 2 is identified in Table 5.

12. The method according to 8 wherein said SNP is one or more SNPS as identified in Table 7.

13. The method according to claim 8 wherein said one or more SNPs comprises or consists of one or more of the following SNPs:
AX-317616757 and AX-317647630;
AX-317616757, AX-317617572 and AX-317645761;
AX-317718855; or combinations thereof, optionally in combination with one or more other SNPs identified in Tables 2, 5 and/or 7.

14. The method according to claim 8 wherein said one or more SNPs comprises or consists of:
AX-317616757, optionally in combination with one or more other SNPs identified in Tables 2, 5 and/or 7.

15. The method according to claim 1 comprising or further comprising detecting one or more nucleotide alterations in one or more genes identified in Table 6.

16. The method according to claim 1 wherein the virus is Tilapia Lake Virus.

17. (canceled)

18. A kit for use in the method of claim 1, the kit comprising or consisting of one or more probes for hybridising to said one or more nucleotide alterations within chromosomes 22 and/or 3.

19. A method of selecting a tilapia for use as broodstock or gene editing wherein the tilapia is selected, in accordance with the method according to claim 1.

20. A fish population obtained following breeding or gene editing a fish to engineer increased resistance, wherein the fish is identified according to claim 1.
